(19) United States
(12) Patent Application Publication
(10) Pub. No.: US 2018/0282407 A1
(43) Pub. Date: Oct. 4, 2018

(54) INHIBITOR OF HEPATITIS C VIRUS INFECTION

(71) Applicant: FUKUSHIMA MEDICAL UNIVERSITY, Fukushima (JP)

(72) Inventors: Hideki CHIBA, Fukushima (JP); Naoki TOMIKAWA, Fukushima (JP); Hiromasa OHIRA, Fukushima (JP); Ken OKAI, Fukushima (JP)

(73) Assignee: Fukushima Medical University, Fukushima (JP)

(21) Appl. No.: 15/765,954

(22) PCT Filed: Oct. 7, 2016
(86) PCT No.: PCT/JP2016/079955

§ 371 Date: Apr. 4, 2018

(30) Foreign Application Priority Data
Oct. 7, 2015 (JP) 2015-199019

(51) Int. Cl. C07K 16/28; A61K 39/29; A61P 31/14

(52) U.S. Cl. CPC C07K 16/28; A61K 39/29; A61P 31/14; C07K 2317/34; C07K 2317/76; C07K 2317/56; C07K 2317/24; C07K 2317/622; C07K 2317/31; C07K 2317/565

(57) ABSTRACT

An inhibitor of HCV infection having a high inhibitory effect on HCV infection wherein the effect is unaffected by genotypes or gene mutations of HCV is developed and provided. The present invention provides an anti-occludin antibody binding to a peptide which consists of a portion of an amino acid sequence constituting a second extracellular domain of occludin and contains the amino acid sequence represented by SEQ ID NO: 1 as an epitope.

### TECHNICAL FIELD

The present invention relates to an antibody having activity for inhibiting hepatitis C virus infection, and an inhibitor of hepatitis C virus infection comprising the same as an active ingredient.

### BACKGROUND ART

Liver cancer is a disease for the fifth most common cause of cancer mortality among different sites of cancer origin in Japan, and more than 30,000 people die of this cancer per year. It is known that 80% thereof is caused by hepatitis C virus (in the present description, also referred to as “HCV”) infection (Non Patent Literature 1).

HCV is an RNA virus classified into the genus Hepacivirus of the family Flaviviridae and is a major virus of viral hepatitis along with hepatitis A virus and hepatitis B virus. This virus is highly host-specific and infects only chimpanzees except humans.

The number of patients infected with HCV is estimated to be 130,000,000 to 170,000,000 people worldwide (Non Patent Literature 2) and approximately 1,900,000 to 2,300,000 people in Japan (Non Patent Literature 3). In addition to these statistical values, it is thought that there exist many asymptomatic virus carriers who are not aware of having HCV infection.

Approximately 70% of HCV-infected patients after early infection reach the state of persistent infection which eventually turns into a refractory disease, i.e. chronic hepatitis C. Thereafter, approximately 60% are known to progress to liver cirrhosis and result in liver cancer in some cases. Liver cancer is known to recur in many patients due to subsequent inflammation at a non-cancerous part even if the cancer is surgically resected.

Triple-drug combination therapy with interferon, an antiviral drug ribavirin, and a NS3/4A protease inhibitor (e.g., simeprevir or vaniprevir) is currently practiced as a method for treating chronic hepatitis C in Japan (Non Patent Literatures 4 and 5). In 2014, a novel method was also introduced for treating chronic hepatitis C/compensated cirrhosis C using an oral NSSA replication complex inhibitor daclatasvir and a NS3/4A protease inhibitor asunaprevir in combination as direct-acting antivirals (DAAs) (Non Patent Literatures 6 to 8).

However, all the aforementioned methods for treating HCV are aimed at suppressing growth after persistent HCV infection. Any vaccine or immunoglobulin effective for preventing infection mediated by blood which is a main route of infection with HCV, for example, vertical transmission such as mother-to-child transmission or horizontal transmission such as accidental injection, has not yet been established. A reason for this is the difficulty in preparing a highly active neutralizing antibody because genes associated with the production of the envelope proteins of HCV are subject to mutations.

Meanwhile, HCV is taken up into host cell hepatocytes through a common entry process of a clathrin-dependent endocytosis mechanism, irrespective of its genotype (Non Patent Literatures 9 and 10). Genes encoding hepatocyte's factors (virus receptors) involved in this intracellular entry of HCV are much less subject to mutations than genes encoding the envelope proteins of HCV. Hence, the virus receptors can serve as promising target substances for the prevention or treatment of HCV.

Many virus receptors have been identified so far. Four factors shown in FIG. 1, i.e., scavenger receptor class B type I (SR-BI) protein (Non Patent Literature 11), a tetraspanin family molecule CD81 protein (Non Patent Literature 12), and tight junction molecules claudin-1 protein (Non Patent Literature 13) and occludin protein (Non Patent Literatures 14 and 15), are known to be factors essential for the establishment of HCV infection. All of these are transmembrane proteins. It has been revealed that among them, the CD81 protein and the occludin protein do not function as HCV entry factors unless they are human- or chimpanzee-derived proteins (Non Patent Literature 15). This has suggested that the CD81 protein and the occludin protein are particularly suitable as target substances for prophylactic agents for HCV.

In fact, an anti-CD81 antibody is known as an inhibitor of HCV infection against human CD81 protein as a target substance (Non Patent Literatures 16 and 17). This antibody has a high inhibitory effect on HCV infection without being influenced by HCV genotypes or mutations. Nonetheless, the administration of the anti-CD81 antibody to chimeric mice with humanized liver (uPA-SCID) showed side effect problems such as elevation in transaminase levels or syncytium formation (Non Patent Literature 18).

Meanwhile, it has been reported as to human occludin protein that: the second extracellular domain is responsible for the species specificity of HCV (Non Patent Literatures 15 and 19); and when the amino acid sequence of the second extracellular domain is compared between human and mouse occludin proteins, 6 different amino acids are present, among which alanine at positions 223 and 224 counted from initiating methionine plays an important role in the HCV sensitivity of the human occludin protein (Non Patent Literature 20). However, there has been no report regarding the direct binding of HCV to the occludin protein. Furthermore, research and development has not been conducted for any inhibitor of HCV infection against the occludin protein as a target substance.

### CITATION LIST

### Non Patent Literature

Non Patent Literature 1: Ministry of Health LaW. Vital Statistics Japan. 2013
Non Patent Literature 2: Shepard C W, et al., 2005, Lancet Infect Dis, 5: 558-567
Non Patent Literature 3: Viral Hepatitis Research Foundation of Japan. Hepatitis C. 2014
Non Patent Literature 4: Hayashi N, et al., 2014, J Hepatol, 61: 219-227
Non Patent Literature 5: Norio Hayashi and Nobuyuki Tanaka, 2014, Kanzo (liver in English), 55: A35
Non Patent Literature 6: Feeney E R & Chung R T., 2014, BMJ, 348: g3308
Non Patent Literature 7: Manns M, et al., 2014, Lancet, 384: 1597-1605
Non Patent Literature 8: Webster D P, et al., 2015, Lancet, 385: 1124-1135
Non Patent Literature 9: Blanchard E, et al., 2006, J Virol, 80: 6964-6972
Non Patent Literature 10: Fofana I, et al., 2014, Antiviral Res, 104: 136-142
Non Patent Literature 11: Scarselli E et al., 2002, EMBO J, 21: 5017-5025
Non Patent Literature 12: Pileri P, et al., 1998, Science, 282: 938-941
Non Patent Literature 13: Evans M J, et al., 2007, Nature 2007; 446: 801-805
Non Patent Literature 14: Liu S, et al., 2009, J Virol, 83: 2011-2014
Non Patent Literature 15: Ploss A, et al., 2009, Nature, 457: 882-886
Non Patent Literature 16: Batosch B, et al., 2003, J. Med. Exp, 197: 633-642
Non Patent Literature 17: Zhang J, et al., 2004, J Virol, 78: 1448-1445
Non Patent Literature 18: Zona L, et al., 2014, Viruses, 6: 875-892
Non Patent Literature 19: Ciesek S, et al., 2011, J Virol, 85: 7613-7621
Non Patent Literature 20: Maria L, et al., 2010, J Virol, 84: 11696-11708

### SUMMARY OF INVENTION

### Technical Problem

An object of the present invention is to develop and provide an inhibitor of HCV infection having a high inhibitory effect on HCV infection wherein the effect is unaffected by genotypes or gene mutations of HCV.

### Solution to Problem

To solve the above-mentioned problems, the present inventors have developed an inhibitor of HCV infection by focusing on the occludin protein which is essential for the establishment of HCV infection. The present inventors have prepared anti-human occludin monoclonal antibodies targeting the second extracellular domain responsible for the species-specific HCV sensitivity of the occludin protein. As a result, all the antibodies have exhibited evident ability to bind to the antigen, but showed no inhibitory effect on HCV infection in an ordinary monolayer culture system. Accordingly, as a result of evaluating the inhibition of HCV infection in a three-dimensional culture system of hepatocytes which is a cellular environment more similar to that in living bodies, an antibody binding to a particular epitope has showed a marked inhibitory effect on HCV infection. Furthermore, this antibody has been not toxic to cells in an amount that exhibits the inhibition of HCV infection. The present invention is based on these findings and provides the following:

(1) An anti-occludin antibody binding to a peptide which consists of a portion of an amino acid sequence constituting a second extracellular domain of occludin and contains the amino acid sequence represented by SEQ ID NO: 1 as an epitope.

(2) The anti-occludin antibody according to (1), wherein the second extracellular domain of occludin consists of any of the following amino acid sequences (a) to (c):

(a) the amino acid sequence represented by SEQ ID NO: 4,
(b) an amino acid sequence derived from the amino acid sequence represented by SEQ ID NO: 4 by deletions, substitutions additions of an amino acid or multiple amino acids, and
(c) an amino acid sequence having 90% or higher amino acid identity to the amino acid sequence represented by SEQ ID NO: 4.

(3) The anti-occludin antibody according to (1) or (2), wherein the peptide consists of the amino acid sequence represented by SEQ ID NO: 6 or 7.

(4) The anti-occludin antibody according to any of (1) to (3), wherein the antibody is a monoclonal antibody.

(5) The anti-occludin antibody according to (4), wherein the monoclonal antibody comprises a heavy chain variable region comprising CDR1 consisting of the amino acid sequence represented by SEQ ID NO: 14, CDR2 consisting of the amino acid sequence represented by SEQ ID NO: 15, and CDR3 consisting of the amino acid sequence represented by SEQ ID NO: 16, and a light chain variable region comprising CDR1 consisting of the amino acid sequence represented by SEQ ID NO: 18, CDR2 consisting of the amino acid sequence represented by SEQ ID NO: 19, and CDR3 consisting of the amino acid sequence represented by SEQ ID NO: 20.

(6) The anti-occludin antibody according to (4) or (5), wherein the monoclonal antibody comprises a heavy chain variable region consisting of the amino acid sequence represented by SEQ ID NO: 13, and a light chain variable region consisting of the amino acid sequence represented by SEQ ID NO: 17.

(7) The anti-occludin antibody according to any of (4) to (6), wherein the antibody is a humanized antibody, a chimeric antibody, a single-chain antibody, or a multispecific antibody.

(8) A fragment of an anti-occludin antibody according to any of (1) to (7), wherein the fragment retains binding activity to the epitope.

(9) An inhibitor of hepatitis C virus infection, comprising an anti-occludin antibody according to at least one of (1) to (7) and/or an anti-occludin antibody fragment according to (8) as an active ingredient.

(10) A vaccine for the inhibition of hepatitis C virus infection, comprising a peptide which consists of a portion of an amino acid sequence constituting a second extracellular domain of occludin and contains the amino acid sequence represented by SEQ ID NO: 1 as an epitope.

(11) The vaccine for the inhibition of hepatitis C virus infection according to (10), wherein the second extracellular domain of occludin consists of any of the following amino acid sequences (a) to (c):

(a) the amino acid sequence represented by SEQ ID NO: 4,
(b) an amino acid sequence derived from the amino acid sequence represented by SEQ ID NO: 4 by deletions substitutions or additions of an amino acid or multiple amino acids, and
(c) an amino acid sequence having 90% or higher amino acid identity to the amino acid sequence represented by SEQ ID NO: 4.

(12) The vaccine for the inhibition of hepatitis C virus infection according to (10) or (11), wherein the peptide consists of 4 to 20 amino acids.

(13) The vaccine for the inhibition of hepatitis C virus infection according to (12), wherein the peptide consists of the amino acid represented by SEQ ID NO: 6 or 7.

The present description incorporates the disclosure of Japanese Patent Application No. 2015-199019 to which the present application claims priority.

### Advantageous Effects of Invention

The antibody and a fragment thereof of the present invention can be used alone as a pharmaceutical or a research reagent, and in addition, can be used as an active ingredient in an inhibitor of HCV infection for the treatment or prevention of hepatitis C.

### BRIEF DESCRIPTION OF DRAWINGS

FIG. 1 is a conceptual diagram showing a route of HCV entry into a host cell and the inhibition of infection by the anti-occludin antibody of the present invention. In the diagram, SR-B1 represents the scavenger receptor class B type I protein, hCD-81 represents the human CD-81 protein, CLDN1 represents the claudin-1 protein, and hOCLN represents the human occludin protein. EC1 and EC2 represent the first extracellular domain and the second extracellular domain, respectively, of occludin. It is considered that HCV sequentially utilizes these virus receptors or coupled receptors thereof to enter hepatocytes through clathrin-dependent endocytosis. The anti-occludin antibody of the present invention binds to EC2 by preceding HCV or competing with HCV and induces endocytosis, thereby inhibiting the endocytosis-mediated intracellular entry of HCV.

FIG. 2 is a diagram showing the infection inhibition rates of 6 clones (C15, C23, C46, C67, C81, and C111) exhibiting at least certain levels of inhibitory effects on HCVpv infection among anti-occludin monoclonal antibody clones obtained in Example 1. The dotted line shows the line indicating the 50% inhibition of infection for con1.

FIG. 3 is a diagram showing a HCVcc infection rate for the 6 clones exhibiting inhibitory effects on HCVpv infection. The HCVcc infection rate is indicated by a relative value when the HCVcc infection rate of a mock group is defined as 1.

FIG. 4 shows ELISA results showing the antigen binding activity of anti-occludin monoclonal antibodies contained in culture supernatants of 5 hybridomas subcloned by limiting dilution. In the diagram, Pre LD represents a culture supernatant before subcloning.

FIG. 5 is a conceptual diagram of a double-chamber culture system used in Example 4.

FIG. 6 is a diagram showing the inhibitory effects on HCVcc infection of 15 subclones of anti-human occludin antibodies using the double-chamber culture system. The infection rate is indicated by a relative value when the infection rate of the mock group is defined as 1. A lower value of the infection rate means a higher inhibitory effect on infection. The antibody of subclone 2 from C67 indicated by an arrow (anti-occludin antibody 67-2; in the present description, often referred to as “67-2 antibody”) exhibited a strong inhibitory effect on HCVcc infection comparable to the positive control CD81 group.

FIG. 7 shows a mean and standard deviation of a HCVcc infection rate for the mock group, the CD81 group, the IgG group, and the anti-occludin antibody 67-2 of FIG. 6. * represents P<0.001 (Mann-Whitney U test).

FIG. 8 is a conceptual diagram of a Matrigel three-dimensional culture system used in Example 5.

FIG. 9 is a diagram showing the inhibitory effect on HCVcc infection of the anti-human occludin antibody 67-2 using the Matrigel three-dimensional culture system. The mean and standard deviation of the HCVcc infection rate is indicated by a relative value when the HCVcc infection rate of the mock group is defined as 1. * represents P<0.001 (Mann-Whitney U test).

FIG. 10 is a diagram showing the cytotoxicity of the anti-occludin antibody 67-2. FIG. 10A shows the cytotoxicity at each concentration of the anti-occludin monoclonal antibody 67-2. The mock group is a negative control without an antibody. The IgG group is normal mouse IgG serving as a negative control antibody having no cytotoxicity. FIG. 10B shows the cytotoxicity of each antibody 2 hours after HCVcc infection (+) and in the absence of HCVcc (−).

FIG. 11A is a conceptual diagram showing the second extracellular domain (EC2) of occludin, and the positions of antigenic peptides ocln1 to ocln3 used in the epitope analysis of the anti-occludin antibody 67-2. In the diagram, the circles depict amino acids constituting EC2, and one circle corresponds to one amino acid. The numerical values with the arrow depict positions in the occludin protein when the position of initiating methionine is defined as 1. The alphabet before the numerical value represents the identity of the amino acid at the position. The filled circles depict the positions of an epitope recognized by the anti-occludin antibody of the present invention. FIG. 11B is a diagram showing results of analyzing the binding activity of the anti-occludin antibody 67-2 against each antigenic peptide shown in FIG. 11A by ELISA. An antigenic peptide (KLH-Ocln peptide) consisting of the second extracellular domain of occludin used in the preparation of the anti-occludin antibody of the present invention was used as a positive control of an antigenic peptide.

FIG. 12 shows results of fluorescent immunostaining of Huh7.5.1 cells, demonstrating changes in the localization of occludin with HCV infection and the administration of the anti-occludin antibody 67-2. FIG. 12A shows a 72 hr fixation group, and FIG. 12B shows a 2 hr fixation group.

FIG. 13 is a diagram showing the inhibitory effect on HCVcc infection of an anti-occludin humanized antibody (2D3 antibody). In the diagram, mock represents a mock group supplemented with only a medium, CD81 represents a CD81 group supplemented with a positive control anti-CD81 antibody, IgG represents an IgG group with normal mouse IgG added to a TBS buffer, and 2D3 represents a 2D3 group supplemented with the anti-occludin humanized antibody 2D3 antibody.

### DESCRIPTION OF EMBODIMENTS

### 1. Anti-Occludin Antibody or Fragment Thereof

### 1-1. Summary

The first aspect of the present invention provides an anti-occludin antibody or a fragment thereof. The anti-occludin antibody or the fragment thereof of the present invention can recognize and bind to a particular epitope on an extracellular domain of a membrane protein occludin. The occludin is a membrane protein necessary for the HCV infection of host cells. The anti-occludin antibody or the fragment thereof of the present invention has an effect of inducing the endocytosis of occludin and inhibiting the cytoplasmic entry of HCV.

### 1-2. Definition

The following terms frequently used in the present description will be defined.

The “hepatitis C virus (HCV)” is a RNA virus belonging to the genus Hepacivirus of the family Flavividae and is known as a major causative virus of non-A, non-B hepatitis (Choo et al., Science, 1989, vol. 244, p. 359-362). This virus is highly host-specific and infects only humans or chimpanzees in principle. The HCV infection usually occurs via blood of transfusion, mother-to-child transmission, accidental injection, or the like. HCV that has entered blood vessels is delivered to the liver through blood flow, then passes through vascular endothelial cells of the hepatic sinusoid, and binds to virus receptors on the membranes of host cell hepatocytes. Thereafter, the virus is taken up into the hepatocytes through endocytosis to establish primary infection. After infection, the genomic RNA of the HCV is released from the HCV virions into the cytoplasms of the hepatocytes. A HCV protein precursor encoded by the genomic RNA is translated. Subsequently, the HCV protein precursor is processed into a core protein, structural proteins (envelope proteins E1 and E2, etc.), and nonstructural (NS) proteins (RNA-dependent RNA polymerase, protease and helicase, etc.) (Moradpour D, et al., 2007, Nat Rev Microbiol, 5: 453-463; and Pawlotsky J M, et al., 2007, Gastroenterology 132: 1979-1998). The nonstructural proteins form a replication complex with factors of the host cell and perform replication of their own genomic RNA. The newly formed HCV genomic RNA forms a complex with the NS5A protein, one of the nonstructural proteins. This complex binds to the core protein to form a nucleocapsid. The nucleocapsid is covered with the envelope proteins in the endoplasmic reticulum within the hepatocyte. Then, a matured virion arrives at the cell membrane through a trans-Golgi-mediated pathway and is released to the outside of the cell. HCV repeats the routes of infection and growth through this series of life cycles.

The “hepatitis C” is a liver disease that is developed by HCV infection. Liver functions decline due to the disruption of hepatocytes caused by inflammation and manifest symptoms such as systemic malaise, fever, anorexia, nausea, and vomiting. It is known that approximately 30% of infected patients heal naturally by eradicating the virus from their bodies through their own immune response, whereas approximately 70% have a chronic condition by persistent infection, eventually progressing to liver cirrhosis or liver cancer.

The “(viral) infection” refers to a series of life cycles of a virus, i.e., a process from the attachment of virions to the cell surface of host cells, subsequent entry into the cells and growth, to be released to the outside of the cells, in the broad sense and refers to an early infection process up to the entry of virions into host cells in the narrow sense. In the present description, the infection is used mainly in the narrow sense, though any of the meanings may be encompassed. Thus, in the present description, the “HCV infection inhibition”, the “inhibition of HCV infection”, etc. refers to the inhibition of the infection in the narrow sense from the attachment of HCV virions to hepatocyte surface to the entry into the cells.

In the present description, the “inhibitor of HCV infection” refers to an agent that inhibits the infection of a subject with HCV by administration to the subject. The inhibitor of HCV infection of the present invention can competitively inhibit the binding of HCV to a HCV receptor on host cell surface by inducing endocytosis through the binding of an antibody or a fragment thereof serving as an active ingredient to the HCV receptor, and thus inhibit the entry of the HCV into the cell.

In the present description, the “host cell” refers to a target cell to be infected with a virus. For HCV, the host cell means a human- or chimpanzee-derived hepatocyte in principle, but also exceptionally encompasses a cell modified to artificially express four cell membrane proteins reportedly essential for the establishment of HCV infection, i.e., a tetraspanin family molecule CD81, SR-BI (scavenger receptor class B type I), and tight junction molecules claudin-1 and occludin, on the cell surface. However, in this case, CD81 and occludin must be derived from a human or a chimpanzee. This is because CD81 and occludin are highly species-specific and do not function as HCV entry factors unless they are human- or chimpanzee-derived.

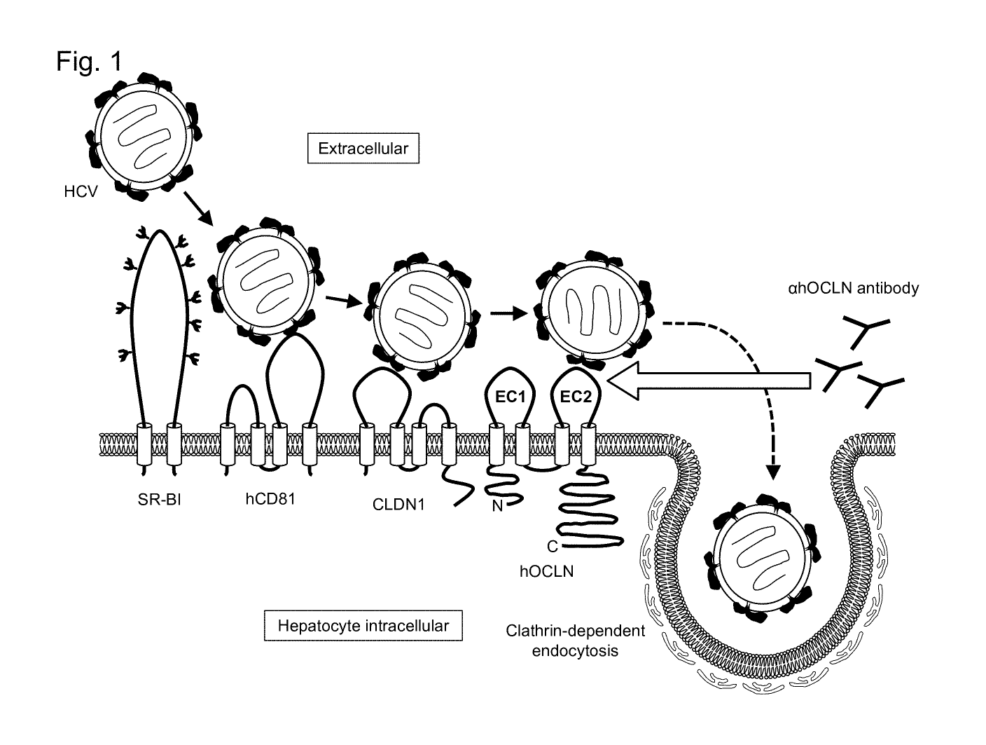

The “occludin protein” (in the present description, often referred to as “occludin”) is a four-pass transmembrane protein identified as a tight junction component involved in cell adhesion. The human occludin protein (hOCLN), as shown in FIG. 1, is constituted by four transmembrane domains, the N-terminal region, the C-terminal region, and the intracellular domain between the second and third transmembrane domains located inside the cell, and the first extracellular domain (EC1) between the first and second transmembrane domains and the second extracellular domain (EC2) between the third and fourth transmembrane domains located outside the cell (Furuse M., et al., 1993, J Cell Biol, 123: 1777-1788). The occludin protein is also a determinant factor of species-specific HCV sensitivity, together with the CD81 protein. Hence, the occludin protein intended in the present description is the human-derived occludin (human occludin) protein consisting of the amino acid sequence represented by SEQ ID NO: 2 or the chimpanzee-derived occludin (chimpanzee occludin) protein consisting of the amino acid sequence represented by SEQ ID NO: 5, in principle.

### 1-3. Configuration

Hereinafter, the anti-occludin antibody and the fragment thereof of the present invention will be specifically described.

### (1) Anti-Occludin Antibody

The “anti-occludin antibody” refers to an antibody that exhibits immune responsiveness to the occludin protein. In the present description, the anti-occludin antibody particularly refers to an antibody that recognizes an amino acid sequence that is contained in the second extracellular domain of the occludin protein and consists of alanine (A)-leucine (L)-cysteine (C)-asparagine (N) shown in SEQ ID NO: 1, as an epitope (in the present description, often referred to as an “ALCN epitope”). In this context, the second extracellular domain of the human occludin protein is constituted by 48 amino acids shown in SEQ ID NO: 4. This corresponds to positions 196 to 243 when the position of initiating methionine is defined as 1 in the amino acid sequence of the full-length human occludin protein shown in SEQ ID NO: 2 (in the present description, the same holds true for the description below unless otherwise specified). The ALCN epitope corresponds to positions 214 to 217.

The second extracellular domain of chimpanzee occludin has an amino acid sequence identical to the second extracellular domain of human occludin. The position of the second extracellular domain and the position of the ALCN epitope in the full-length chimpanzee occludin are the same as in the human occludin. Thus, the anti-occludin antibody in the present description is capable of recognizing and binding to the human occludin and the chimpanzee occludin.

The anti-occludin antibody of the present invention can be any antibody that exhibits immune responsiveness to an antigenic peptide comprising a portion of an amino acid sequence constituting the second extracellular domain of occludin. This antigenic peptide comprises the ALCN epitope. As mentioned above, the second extracellular domain is constituted by the amino acid sequence represented by SEQ ID NO: 4 in principle, but may be any of other amino acid sequences that retain the ALCN epitope. Examples thereof include an amino acid sequence derived from the amino acid sequence represented by SEQ ID NO: 4 by deletions, substitutions or additions of an amino acid or multiple amino acids, and an amino acid sequence having 90% or higher amino acid identity to the amino acid sequence represented by SEQ ID NO: 4.

In the present description, the term “multiple” refers to, for example, 2 to 20, 2 to 15, 2 to 10, 2 to 7, 2 to 5, 2 to 4, or 2 or 3. The “amino acid identity” refers to the ratio (%) of the number of matched amino acid residues to the total number of amino acid residues between two amino acid sequences to be compared. Specifically, two amino acid sequences are aligned, and a gap is appropriately inserted to one or both of the amino acid sequences, if necessary. The total number of amino acid residues is counted with one gap regarded as one amino acid residue. The alignment of the amino acid sequences can be performed, for example, by using a known program such as Blast, FASTA, or ClustalW (Karlin, S. et al., 1993, Proc. Natl. Acad. Sci. USA, 90: 5873-5877; Altschul, S. F. et al., 1990, J. Mol. Biol., 215: 403-410; and Pearson, W. R. et al., 1988, Proc. Natl. Acad. Sci. USA, 85: 2444-2448). When the total number of amino acid residues differs between the two amino acid sequences to be compared, the total number of amino acid residues of a longer sequence is adopted. The amino acid identity is calculated by dividing the number of identical amino acid residues by the total number of amino acid residues when the two amino acid sequences to be compared are aligned at the highest degree of amino acid match.

In the present description, the “(amino acid) substitution” refers to substitution within a conservative amino acid group having similarity in properties such as charge, side chain, polarity, and aromaticity among 20 types of amino acids constituting a natural protein. Examples thereof include substitution within an uncharged polar amino acid group having a low polar side chain (Gly, Asn, Gln, Ser, Thr, Cys, and Tyr), a branched-chain amino acid group (Leu, Val, and Ile), a neutral amino acid group (Gly, Ile, Val, Leu, Ala, Met, and Pro), a neutral amino acid group having a hydrophilic side chain (Asn, Gln, Thr, Ser, Tyr, and Cys), an acidic amino acid group (Asp and Glu), a basic amino acid group (Arg, Lys, and His), or an aromatic amino acid group (Phe, Tyr, and Trp). Amino acid substitution within any of these groups is preferred because the amino acid substitution rarely changes polypeptide properties.

The antigenic peptide that is recognized by the anti-occludin antibody of the present invention preferably consists only of a region consisting of a portion of the amino acid sequence constituting the second extracellular domain of occludin (hereinafter, referred to as an “occludin second extracellular domain region”), but may comprise a non-occludin second extracellular domain region consisting of any of other peptides or amino acids. Examples of such a non-occludin second extracellular domain region include a C-terminal partial region of the first transmembrane domain of occludin. The non-occludin second extracellular domain region is placed on the N-terminal or C-terminal side, or both, of the occludin second extracellular domain region.

The amino acid length of the antigenic peptide that is recognized by the anti-occludin antibody of the present invention is not particularly limited. However, the length of the occludin second extracellular domain region is 4 amino acids constituted only by the ALCN epitope at the shortest and is a length of the full-length of the second extracellular domain minus 1 amino acid, i.e., 47 amino acids for the second extracellular domain of human occludin shown in SEQ ID NO: 4, at the longest. Thus, when the antigenic peptide is constituted only by the occludin second extracellular domain region, the length thereof falls within the range of 4 amino acids (ALCN epitope) to 47 amino acids. Usually, a length on the order of 5 to 40 amino acids, 7 to 30 amino acids, or 8 to 25 amino acids suffices.

The organism species from which the anti-occludin antibody of the present invention is derived is not particularly limited. A bird- or mammal-derived antibody is preferred. Examples thereof include chickens, ostriches, mice, rats, guinea pigs, rabbits, goats, donkeys, sheep, camels, horses, and humans.

The anti-occludin antibody of the present invention may be a monoclonal antibody or a polyclonal antibody as long as the antibody recognizes the ALCN epitope as an epitope and exhibits immune responsiveness thereto. A monoclonal antibody having a stable antibody titer is preferred. In the present description, the “polyclonal antibody” refers to a mixture of a plurality of different immunoglobulins that can specifically bind to the antigen occludin and recognize it. In the present description, the “monoclonal antibody” refers to a single immunoglobulin that comprises framework regions (hereinafter, referred to as “FRs”) and complementarity determining regions (hereinafter, referred to as “CDRs”) and can specifically bind to the antigen occludin and recognize it, or a recombinant antibody or a synthetic antibody containing at least one set of a light chain variable region (V_{L} region) and a heavy chain variable region (V_{H} region) contained in the immunoglobulin.

When the anti-occludin antibody is constituted by an immunoglobulin molecule, the immunoglobulin can be of any class (e.g., IgG, IgE, IgM, IgA, IgD and IgY) or any subclass (e.g., IgG1, IgG2, IgG3, IgG4, IgA1 and IgA2).

The anti-occludin monoclonal antibody of the present invention specifically binds to the ALCN epitope shown in SEQ ID NO: 1 on the occludin protein. Specific examples of such a monoclonal antibody include mouse anti-human occludin monoclonal antibody clone 67-2 described in Example 4 mentioned later. The heavy chain variable region of the 67-2 antibody consists of the amino acid sequence represented by SEQ ID NO: 13, and the light chain variable region consists of the amino acid sequence represented by SEQ ID NO: 17. According to the Kabat rule (Kabat E. A., et al., 1991, Sequences of proteins of immunological interest, Vol. 1, eds. 5, NIH publication), CDR1 in the heavy chain variable region of the 67-2 antibody consists of the amino acid sequence represented by SEQ ID NO: 14, CDR2 consists of the amino acid sequence represented by SEQ ID NO: 15, and CDR3 consists of the amino acid sequence represented by SEQ ID NO: 16. Also, CDR1 in the light chain variable region consists of the amino acid sequence represented by SEQ ID NO: 18, CDR2 consists of the amino acid sequence represented by SEQ ID NO: 19, and CDR3 consists of the amino acid sequence represented by SEQ ID NO: 20.

Examples of a nucleic acid (nucleotide) encoding the amino acid sequence represented by SEQ ID NO: 13 which corresponds to the heavy chain variable region of the 67-2 antibody include a nucleic acid consisting of the nucleotide sequence represented by SEQ ID NO: 21. Furthermore, examples of a nucleic acid encoding the amino acid sequence represented by SEQ ID NO: 17 which corresponds to the light chain variable region of the 67-2 antibody include a nucleic acid consisting of the nucleotide sequence represented by SEQ ID NO: 25. Additionally, examples of nucleotide sequences respectively encoding CDR1, CDR2, and CDR3 of the heavy chain variable region in the 67-2 antibody include nucleic acids consisting of the nucleotide sequences represented by SEQ ID NOs: 22, 23, and 24, respectively. Examples of nucleotide sequences respectively encoding CDR1, CDR2, and CDR3 of the light chain variable region in the 67-2 antibody further include nucleic acids consisting of the nucleotide sequences represented by SEQ ID NOs: 26, 27, and 28, respectively.

The “recombinant antibody” refers to a chimeric antibody or a humanized antibody. The “chimeric antibody” is an antibody prepared by combining the amino acid sequences of antibodies derived from different animals and is an antibody in which the constant regions (C regions) of a certain antibody are replaced with the C regions of another antibody. The chimeric antibody includes, for example, an antibody in which the C regions of a mouse monoclonal antibody are replaced with the C regions of a human antibody. Specific examples thereof is an antibody prepared by replacing the heavy chain variable region consisting of the amino acid sequence represented by SEQ ID NO: 13 in the aforementioned 67-2 antibody with the heavy chain variable region of a human antibody, and replacing the light chain variable region consisting of the amino acid sequence represented by SEQ ID NO: 17 in the 67-2 antibody with the light chain variable region of the human antibody. This can reduce immune response to the antibody in the human body. The “humanized antibody” is a mosaic antibody in which CDRs in a human antibody are replaced with CDRs of a nonhuman mammal-derived antibody. A variable region (V region) of an immunoglobulin molecule is constituted by linking four FRs (FR1, FR2, FR3 and FR4) and three CDRs (CDR1, CDR2 and CDR3) in order of FR1-CDR1-FR2-CDR2-FR3-CDR3-FR4 from the N terminus. Among them, FRs are relatively conserved regions constituting the backbone of the variable region, and CDRs directly contribute to the antigen binding specificity of the antibody. The humanized antibody can be constructed, for example, as a human antibody with antigen binding specificity inherited from a mouse anti-occludin antibody by replacing a set of light chain or heavy chain CDRs (CDR1, CDR2, and CDR3) in a human antibody against an arbitrary antigen with a set of light chain or heavy chain CDRs (CDR1, CDR2 and CDR3), respectively, of a mouse-derived anti-occludin antibody. Specific examples thereof include an antibody prepared by replacing heavy chain CDR1, CDR2, and CDR3 of a human antibody with heavy chain-derived CDR1 consisting of the amino acid sequence represented by SEQ ID NO: 14, CDR2 consisting of the amino acid sequence represented by SEQ ID NO: 15, and CDR3 consisting of the amino acid sequence represented by SEQ ID NO: 16, respectively, in the aforementioned 67-2 antibody, and replacing light chain CDR1, CDR2, and CDR3 of the human antibody with light chain-derived CDR1 consisting of the amino acid sequence represented by SEQ ID NO: 18, CDR2 consisting of the amino acid sequence represented by SEQ ID NO: 19, and CDR3 consisting of the amino acid sequence represented by SEQ ID NO: 20, respectively, in the aforementioned 67-2 antibody. Such a humanized antibody has human antibody-derived regions except for CDRs and can therefore reduce immune response to the antibody in the human body, more than the chimeric antibody.

The “synthetic antibody” refers to an antibody synthesized chemically or by use of a recombinant DNA method. Examples thereof include an antibody newly synthesized by a recombinant DNA method. Specific examples thereof include scFv (single chain Fragment of variable region), diabody, triabody and tetrabody. In an immunoglobulin molecule, a set of variable regions (light chain variable region V_{L} and heavy chain variable region V_{H}) forming a functional antigen binding site are located on separate polypeptide chains, i.e., a light chain and a heavy chain. The scFv is a synthetic antibody with a molecular weight of approximately 35 kDa or smaller having a structure where V_{L} and V_{H} in an immunoglobulin molecule are linked via a flexible linker with a sufficient length and contained in one polypeptide chain. A set of variable regions within the scFv can self-assemble with each other to form one functional antigen binding site. The scFv can be obtained by integrating recombinant DNA encoding it into a vector by use of a known technique, followed by expression. The diabody is a molecule having a structure based on the dimeric structure of scFv (Holliger et al., 1993, Proc. Natl. Acad. Sci. USA 90: 6444-6448). For example, when the length of the linker described above is shorter than approximately 12 amino acid residues, the two variable regions within scFv cannot self-assemble. However, two scFvs can form diabody through interaction so that V_{L} of one of the scFvs can assemble with V_{H} of the other scFv to form two functional antigen binding sites. Moreover, a disulfide bond can also be formed between the two scFvs by the addition of cysteine residues to the C termini of the scFvs to form a stable diabody. Thus, the diabody is a divalent antibody fragment. The triabody and the tetrabody are trivalent and tetravalent antibodies, respectively, having trimeric and tetrameric structures based on the scFv structure, as in the diabody. The diabody, the triabody, and the tetrabody may be a multispecific antibody. The “multispecific antibody” refers to a multivalent antibody, i.e., an antibody having a plurality of antigen binding sites in one molecule, wherein each antigen binding site binds to different epitopes. Examples thereof include a bispecific antibody wherein the antibody is a diabody and each antigen binding site thereof binds to different epitopes. Specifically, the multispecific antibody of the anti-occludin antibody of the present invention includes, for example, a diabody in which one of the antigen binding sites binds to the ALCN epitope, and the other antigen binding site binds to an epitope, other than the ALCN epitope, on the extracellular domain of occludin.

The anti-occludin antibody of the present invention may be modified. In this context, the “modification” includes a functional modification necessary for antigen-specific binding activity, such as glycosylation, and a modification with a label necessary for antibody detection.

The modification of the anti-occludin antibody by glycosylation is performed in order to adjust the affinity of the anti-occludin antibody for the occludin second extracellular domain region which is a target region. Specific examples thereof include a modification to remove a glycosylation site by introducing substitutions to amino acid residues constituting the glycosylation in FR of the anti-occludin antibody so that the glycosylation at the site is lost.

Examples of the label for the anti-occludin antibody include labels with fluorescent dyes (FITC, rhodamine, Texas Red, Cy3, and Cy5), fluorescent proteins (e.g., PE, APC, and GFP), enzymes (e.g., horseradish peroxidase, alkaline phosphatase, and glucose oxidase), radioisotopes (e.g., ³H, ¹⁴C, and ³⁵S) and biotin or (strept)avidin.

The anti-occludin antibody of the present invention has a dissociation constant of preferably 10⁻⁷ M or less for occludin. The anti-occludin antibody of the present invention preferably has high affinity of 10⁻⁸ M or less. The dissociation constant is more preferably 10⁻⁹ M or less, particularly preferably 10⁻¹⁰ M or less. The dissociation constant can be measured by use of a technique known in the art. The dissociation constant may be measured using, for example, rate evaluation kit software from Biacore system (GE Healthcare Japan Corp.).

The inhibitor of HCV infection of the present invention may comprise a single anti-occludin antibody or a plurality of anti-occludin antibodies. In the case of comprising a plurality of anti-occludin antibodies, at least one of them needs to be an anti-occludin antibody of the present invention, i.e., an antibody that exhibits immune responsiveness to the ALCN epitope. However, the other anti-occludin antibody (or antibodies) may each be an antibody that recognizes an epitope other than the ALCN epitope on the extracellular domain of occludin.

### (2) Fragment Thereof

In the present description, the “fragment thereof” refers to an antibody fragment that consists of a portion of the anti-occludin antibody and exhibits immune responsiveness to the ALCN epitope, as in the anti-occludin antibody. The fragment includes, for example, Fab, F(ab′)₂, and Fab′.

The Fab is an antibody fragment resulting from the papain cleavage of an IgG molecule at a site closer to the N terminus than the disulfide bond of the hinge and is constituted by C_{H}1, which is adjacent to V_{H}, among three domains (C_{H}1, C_{H}2, and C_{H}3) constituting a H chain constant region (heavy chain constant region; hereinafter, referred to as C_{H}), V_{H}, and a full-length L chain.

The F(ab′)₂ is a dimer of Fab′ resulting from the pepsin cleavage of an IgG molecule at a site closer to the C terminus than the disulfide bond of the hinge. The Fab′ has a structure substantially equivalent to Fab, though its H chain is slightly longer than that of the Fab because of containing the hinge. The Fab′ can be obtained by reducing F(ab′)₂ under mild conditions and cleaving the disulfide linkage of the hinge region. All of these antibody fragments contain the antigen binding site and therefore have the ability to specifically bind to the antigen epitope (in the present description, the ALCN epitope).

### 1-4. Preparation of Anti-Occludin Antibody

The anti-occludin antibody of the present invention can be obtained by a routine method in the art. Provided that the amino acid sequence of the monoclonal antibody is known, the anti-occludin antibody can also be prepared by use of a chemical synthesis method or a recombinant DNA technique on the basis of this amino acid sequence. Moreover, the monoclonal antibody can also be obtained from a hybridoma producing the antibody.

Hereinafter, methods for preparing the anti-occludin antibody of the present invention, i.e., the anti-occludin polyclonal antibody and the anti-occludin monoclonal antibody, and a hybridoma producing the anti-occludin monoclonal antibody will be described with reference to specific examples.

### (1) Preparation of Immunogen

The antigenic peptide as an immunogen is prepared. Examples of the antigenic peptide that may be used as an immunogen for the anti-occludin antibody of the present invention include a peptide which is a portion of the second extracellular domain of human occludin consisting of the amino acid sequence represented by SEQ ID NO: 4 and contains the ALCN epitope consisting of the amino acid sequence represented by SEQ ID NO: 1 (hereinafter, referred to as an “antigenic occludin peptide”).

The antigenic occludin peptide can be prepared by use of, for example, a chemical synthesis method or a DNA recombination technique.

### (1-1) Chemical Synthesis

In the case of preparing the antigenic occludin peptide by use of the chemical synthesis method, the antigenic occludin peptide can be chemically synthesized by an approach known in the art, for example, a solid-phase peptide synthesis method, on the basis of information on, for example, the amino acid sequence of SEQ ID NO: 4. The peptide synthesis can also be outsourced to a manufacturer.

### (1-2) DNA Recombination Technique

In the case of preparing the antigenic occludin peptide by use of the DNA recombination technique, cDNA encoding the antigenic occludin peptide (antigenic occludin peptide cDNA) can be integrated into an appropriate expression system and expressed.

For the antigenic occludin peptide, a nucleotide of an appropriate length comprising a coding region of the ALCN epitope is selected on the basis of information on, for example, the nucleotide sequence represented by SEQ ID NO: 3 encoding the second extracellular domain in the occludin gene. For example, when the antigenic occludin peptide consists of 15 amino acids, a nucleotide sequence region encoding the amino acid sequence is selected from SEQ ID NO: 3, and the antigenic occludin peptide cDNA is chemically synthesized on the basis of information on the sequence consisting of 45 bases. The nucleotide synthesis may be outsourced to a manufacturer. Alternatively, in the case of having cDNA encoding occludin (occludin cDNA) or cDNA encoding the second extracellular domain of occludin (occludin second extracellular domain cDNA), the antigenic occludin peptide cDNA may be prepared by a nucleic acid amplification method such as PCR using the occludin cDNA or the occludin second extracellular domain cDNA as a template and a primer pair appropriately designed to obtain the antigenic occludin peptide cDNA as an amplification product. In this case, an appropriate restriction site for the cloning of the antigenic occludin peptide cDNA or a tag sequence (FLAG, HA, His, myc, GFP, etc.) for protein purification may be introduced to the 5′ end of a primer.

Next, the antigenic occludin peptide cDNA thus obtained is integrated into an appropriate expression system and expressed. The expression system is preferably an expression vector that utilizes a plasmid or a virus. An expression vector capable of replicating in host cells is used. As a host cell, for example, Escherichia coli, Bacillus subtilis, yeast (e.g., Saccharomyces cerevisiae or Schizosaccharomyces pombe), insect cells (e.g., Sf cells), or animal cells (e.g., HEK293, HeLa, COS, CHO, and BHK) can be used. The expression vector can usually comprise, for example, a promoter, a terminator, an enhancer, a polyadenylation signal, a replication origin, and a selection marker as regulatory elements. Also, the expression vector used may have a multicloning site for the cloning of the cDNA fragment of interest, or a tag sequence on the 5′-terminal or 3′-terminal side of an insertion site for the cDNA fragment such that the antigenic occludin peptide is expressed as a fusion polypeptide with a labeling peptide (tag) to facilitate purification. Furthermore, the expression vector used may have a sequence encoding a secretory signal sequence on the 5′-terminal side of the insertion site. This allows an expressed mature polypeptide to be secreted to the outside of cells. Such expression vectors or other expression systems are commercially available as useful products from Takara Bio Inc., Daiichi Pure Chemicals Co., Ltd., Agilent Technologies, Inc., Merck & Co., Inc., Qiagen N. V., Promega Corp., Roche Diagnostics K.K., Thermo Fisher Scientific Inc., and GE Healthcare Japan Corp., etc. Hence, these products may be used.

In this way, the expression system encoding the antigenic occludin peptide cDNA (e.g., an antigenic occludin peptide expression vector) can be obtained.

The obtained antigenic occludin peptide expression system is introduced into host cells, and if necessary, appropriate expression induction treatment can be performed to express the antigenic peptide serving as an immunogen. The expression may utilize a cell-free translation system. The method for introducing the antigenic occludin peptide expression vector into host cells can conform to a known method for introducing DNA into each host cell and is not particularly limited. When the host is, for example, Escherichia coli, examples thereof include a heat shock method, a calcium ion method, and electroporation. When the host is animal cells, examples thereof include a lipofection method, electroporation, a calcium phosphate method, and a DEAE-dextran method. Alternatively, a commercially available transfection reagent such as Lipofectamine 2000 (Thermo Fisher Scientific Inc.) may be used. Transformants for antigenic occludin peptide expression can be obtained by the operation mentioned above.

In order to allow the transformants for antigenic occludin peptide expression to express the antigenic occludin peptide, gene expression induction treatment unique to the antigenic occludin peptide expression system contained in the transformants can be performed. When the antigenic occludin peptide expression system is, for example, a system consisting of a lac repressor gene and a lac operator, the expression of the antigenic occludin peptide encoded within the system can be induced by IPTG (isopropyl-1-thio-β-D-Galactoside) treatment.

When the antigenic occludin peptide is secreted to the outside of cells, the antigenic occludin peptide expressed within the transformants by the expression induction treatment is recovered from the culture supernatant. When the antigenic occludin peptide is accumulated within cells, the cells are disrupted, followed by extraction. Then, the antigenic occludin peptide can be isolated and purified by use of a common protein purification method. When the antigenic occludin peptide is expressed as a fusion peptide with a labeling peptide (tag), for example, affinity chromatography suitable for each labeling peptide can be used. When the antigenic occludin peptide is expressed without a labeling peptide, for example, an ammonium sulfate precipitation method, gel chromatography, ion-exchange chromatography, hydrophobic chromatography, or isoelectric chromatography can be used. Alternatively, two or more of the purification methods described above can be appropriately combined for isolation and purification.

Whether or not the antigenic occludin peptide of interest can be recovered finally can be confirmed by SDS-PAGE or the like.

For details in the method for preparing the antigenic occludin peptide, see protocols in the art, for example, Green, M. R. and Sambrook, J., 2012, Molecular Cloning: A Laboratory Manual Fourth Ed., Cold Spring Harbor Laboratory Press, Cold Spring Harbor, N.Y.

### (2) Immunization and Preparation of Anti-Occludin Polyclonal Antibody

Subsequently, the obtained occludin polypeptide fragment is used as an immunogen to prepare an anti-occludin polyclonal antibody specifically recognizing the polypeptide.

First, the occludin polypeptide fragment is dissolved in a buffer solution to prepare an immunogen solution. In this operation, an adjuvant may be added thereto, if necessary, in order to effectively perform immunization. For example, a Freund complete adjuvant (FCA), a Freund incomplete adjuvant (FIA), an aluminum hydroxide gel, pertussis vaccine, Titer Max Gold (Vaxel Inc.), and GERBU adjuvant (GERBU Biotechnik GmbH) can be used alone or as a mixture as the adjuvant.

Next, the prepared immunogen solution is administered to a mammal for immunization. The animal for use in immunization is not particularly limited. For example, a mouse, a rat, a hamster, a guinea pig, a rabbit, a goat, a donkey, sheep, a camel, or a horse can be used. In the present invention, a method using a mouse will be specifically described below as an example. The strain of the mouse is not particularly limited. For example, an inbred mouse BALB/c can be used in the immunization.

Examples of the method for administering the immunogen solution include, but are not limited to, subcutaneous injection using FIA or FCA, intraperitoneal injection using FIA, and intravenous injection using physiological saline. The administration may be performed by intracutaneous injection or intramuscular injection. A single dose of the immunogen is appropriately determined depending on the type and size of the animal to be immunized, an administration route, etc. For a mouse, approximately 50 to 200 μg can usually be administered to a 4- to 10-week-old individual. The immunization interval is not particularly limited and is several days to several weeks, preferably 1 to 4 weeks. Booster is preferably performed after initial immunization, and the number of booster shots is 2 to 6, preferably 3 or 4. Preferably, blood is collected from the immunized mouse after initial immunization, and the antibody titer in serum is measured by ELISA or the like. Provided that sufficient elevation in the antibody titer is confirmed, the immunogen solution is intravenously or intraperitoneally injected to the mouse for final immunization. Preferably, no adjuvant is used for the final immunization. 3 to 10 days after the final immunization, blood is collected from the immunized mouse, and the serum can be treated in accordance with a known method (Antibodies: A Laboratory Manual Cold Spring Harbor Laboratory, 1988) to obtain the anti-occludin polyclonal antibody.

### (3) Preparation of Hybridoma Producing Anti-Occludin Monoclonal Antibody

The anti-occludin monoclonal antibody can be prepared by a routine method in the art. The anti-occludin monoclonal antibody can be prepared, for example, by fusing antibody-producing cells obtained from the immunized animal described above with antibody-producing cells by a cell fusion method and then selecting a hybridoma clone producing the anti-occludin monoclonal antibody from the obtained hybridomas (Kohler G. & Milstein C., 1975, Nature, 256: 495-497). Hereinafter, the method for preparing the hybridoma producing the anti-occludin monoclonal antibody will be described with reference to specific examples.

First, antibody-producing cells are collected from the immunized mouse in the paragraph (2). The collection is preferably performed 2 to 5 days after the day of final immunization. Examples of the antibody-producing cells include spleen cells, lymph node cells, and peripheral blood cells. Spleen cells or local lymph node cells are preferred. The method for collecting the antibody-producing cells from the mouse can be performed according to a technique known in the art.

Subsequently, the antibody-producing cells are fused with myeloma cells to prepare hybridomas.

The myeloma cells for use in cell fusion are not particularly limited as long as the myeloma cells are a generally available mouse-derived established cell line and are capable of growing in vitro. For conveniently screening hybridomas in a step mentioned later, it is preferred that the myeloma cells should have drug selectivity and have the property of being unable to survive in an unfused state in a selective medium and being able to survive only in a state fused with the antibody-producing cells. For example, P3 (P3x63Ag8.653) (Kearney J. F. et al., 1979, J. Immunol., 123: 1548-1550), an 8-azaguanine-resistant mouse (BALB/c-derived) myeloma cell line P3-X63Ag8-U1 (P3-U1) (Yelton D. E. et al., 1978, Curr. Top. Microbiol. Immunol., 81: 1-7), P3-X63-Ag8 (X63), P3/NS1/1-Ag4-1 (NS1), NS-1 (Kohler G. et al., 1976, Eur. J. Immunol., 6: 511-519), MPC-11 (Margulies D. H. et al., 1976, Cell, 8: 405-415), SP2/0 (Shulman M. et al., 1978, Nature, 276: 269-270), FO (de St. Groth S. F. et al., 1980, J. Immunol. Methods, 35: 1-21), S194 (Trowbridge I. S. 1978, J. Exp. Med., 148: 313-323), or R210 (Galfre G. et al., 1979, Nature, 277: 131-133) is preferably used. These cell lines are available from RIKEN BioResource Center, ATCC (American Type Culture Collection) or ECACC (European Collection of Cell Cultures). Their culture and subculture can conform to known methods (e.g., Antibodies: A Laboratory Manual Cold Spring Harbor Laboratory, 1988, Selected Methods in Cellular Immunology W. H. Freeman and Company, 1980). Examples of the selective medium include HAT medium (RPMI1640 medium supplemented with 100 units/mL of penicillin, 100 μg/mL streptomycin, 10% fetal bovine serum (FBS), 10⁻⁴ M hypoxanthine, 1.5×10⁻⁵ M thymidine and 4×10⁻⁷M aminopterin).

For the cell fusion of the antibody-producing cells with the myeloma cells, the spleen cells and the myeloma cells washed can be mixed at a myeloma cell/antibody-producing cell mixing ratio of 1:1 to 1:10 in a medium for animal cell culture such as MEM, DMEM, or RPMI-1640 medium, or a commercially available medium for cloning or cell fusion (preferably, serum-free) and contacted with each other at 30 to 37° C. for 1 to 15 minutes in the presence of a cell fusion promoter. For example, polyethylene glycol (hereinafter, referred to as “PEG”) with an average molecular weight of 1,500 to 4,000 Da can be used at a concentration of approximately 10 to 80% as the cell fusion promoter. In addition, a fusion promoter or a fusion virus, such as polyvinyl alcohol or hemagglutinating virus of Japan can also be used. Usually, PEG with an average molecular weight of 1,500 Da is preferably used. In order to enhance fusion efficiency, an auxiliary such as dimethyl sulfoxide may be used in combination therewith, if necessary. Alternatively, the antibody-producing cells may be fused with the myeloma cells using a commercially available cell fusion apparatus that utilizes electric stimulation (e.g., electroporation) (Nature, 1977, Vol. 266, 550-552).

After the cell fusion treatment, the cells are washed with the medium (e.g., RPMI1640 medium) used in the fusion of the myeloma cells. Then, a cell suspension is prepared. Subsequently, the cell suspension is appropriately diluted with, for example, FBS-containing RPMI1640 medium and then placed at 1×10⁴ cells/well on a 96-well plate. The selective medium is added to each well. Subsequently, the cells can be cultured with the selective medium appropriately replaced with a fresh one. The culture temperature is 20 to 40° C., preferably approximately 37° C. When the myeloma cells are a HGPRT-deficient line or a thymidine kinase (TK)-deficient line, only hybridomas of the antibody-producing cells and the myeloma cells can selectively survive and grow by use of a selective medium containing hypoxanthine, aminopterin and thymidine (HAT medium). Hence, cells grown approximately 10 days after the start of culture in the selective medium can be selected as hybridomas.

Next, the culture supernatant of the grown hybridomas is screened for whether or not to contain the anti-occludin monoclonal antibody of interest. The hybridoma screening can be performed, for example, by collecting a portion of the culture supernatant contained in the wells for hybridoma culture, followed by screening by enzyme immunoassay (ELISA, etc.), radioimmunoassay (RIA), or the like using binding activity against the occludin polypeptide fragment used as an immunogen as an index. In order to further obtain hybridomas stably producing the monoclonal antibody, the antibody-producing hybridomas are cloned. The cloning method can be performed by an ordinary method such as limiting dilution or fluorescence-activated cell sorting and is not particularly limited. Hybridomas which are anti-occludin monoclonal antibody-producing cells can be established finally by combining these screening and cloning methods.

Cross reactivity may be tested, if necessary. Specifically, binding activity against the first extracellular domain of occludin or other tight junction proteins is examined to select only a hybridoma producing an antibody that exhibits acceptable cross reactivity. The acceptable cross reactivity means the nonspecific binding activity of the monoclonal antibody to a negligible extent for the intended purpose.

### (4) Recovery of Anti-Occludin Monoclonal Antibody

The anti-occludin monoclonal antibody can be recovered by a conventional technique. For example, an ordinary cell culture method or ascitic fluid formation method can be adopted as a method for recovery from the established hybridomas. In the cell culture method, the anti-occludin monoclonal antibody-producing hybridomas are cultured, for example, at 37° C. at a 5% CO₂ concentration for 2 to 10 days, in an animal cell culture medium such as RPMI-1640 medium containing 10% FBS, MEM medium or serum-free medium, and the antibody is obtained from the culture supernatant. In the ascitic fluid formation method, approximately 10,000,000 anti-occludin monoclonal antibody-producing hybridomas are intraperitoneally administered to an animal of the same species as in the mammal from which the myeloma cells are derived (in the case of the paragraph (3), a mouse) so that the hybridomas are allowed to grow at a large scale. 1 to 2 weeks later, ascitic fluid or serum can be collected for recovery.

When antibody purification is necessary, the antibody can be purified by appropriately using a known method. The antibody can be purified by use of, for example, ion-exchange chromatography, affinity chromatography using protein A, protein G or the like, gel chromatography, or an ammonium sulfate precipitation method.

### 2. Inhibitor of Hepatitis C Virus Infection

### 2-1. Summary

The first aspect of the present invention provides an inhibitor of hepatitis C virus (HCV) infection. The inhibitor of HCV infection of the present aspect comprises an anti-occludin antibody or a fragment thereof as an active ingredient. The anti-occludin antibody or the fragment thereof has an effect of inducing the endocytosis of a membrane protein occludin acting as a stepping stone to the HCV infection of host cells, and inhibiting the cytoplasmic entry of HCV.

### 2-2. Configuration

The inhibitor of HCV infection of the present invention contains the active ingredient as an essential constituent and a pharmaceutically acceptable carrier or an additional drug as an optional component. The inhibitor of HCV infection of the present invention may be constituted only by the active ingredient. However, for facilitating formulation and maintaining the pharmacological effect and/or dosage form of the active ingredient, it is preferred that the inhibitor of HCV infection should be constituted as a pharmaceutical composition containing a pharmaceutically acceptable carrier mentioned later.

### 2-2-1. Constituent

Hereinafter, each component constituting the inhibitor of HCV infection of the present invention will be specifically described.

### (1) Active Ingredient

The active ingredient in the inhibitor of HCV infection of the present invention is the anti-occludin antibody and/or the fragment thereof described in the first aspect. Their configurations are already mentioned in detail in the first aspect, so that the specific description thereof will be omitted here.

### (2) Pharmaceutically Acceptable Carrier

The “pharmaceutically acceptable carrier” refers to a solvent and/or an additive that can be usually used in the field of pharmaceutical technology and is not or hardly harmful to living bodies.

Examples of the pharmaceutically acceptable solvent include water, ethanol, propylene glycol, ethoxylated isostearyl alcohol, polyoxylated isostearyl alcohol, and polyoxyethylene sorbitan fatty acid esters. These are desirably sterilized and preferably adjusted to be isotonic to blood, if necessary.

Examples of the pharmaceutically acceptable additive include excipients, binders, disintegrants, fillers, emulsifiers, flow modulators, and lubricants.

Examples of the excipients include sugars such as monosaccharides, disaccharides, cyclodextrin and polysaccharides (more specifically including, but not limited to, glucose, sucrose, lactose, raffinose, mannitol, sorbitol, inositol, dextrin, maltodextrin, starch and cellulose), metal salts (e.g., sodium chloride, sodium phosphate or calcium phosphate, calcium sulfate, magnesium sulfate, and calcium carbonate), citric acid, tartaric acid, glycine, low-, medium- or high-molecular-weight polyethylene glycol (PEG), Pluronic, kaolin, silicic acid and combinations thereof.

Examples of the binders include starch pastes using starch of corn, wheat, rice, or potato, simple syrup, glucose solutions, gelatin, tragacanth, methylcellulose, hydroxypropylmethylcellulose, carboxymethylcellulose sodium, shellac and/or polyvinylpyrrolidone.

Examples of the disintegrants include the starch described above, lactose, carboxymethyl starch, crosslinked polyvinylpyrrolidone, agar, laminaran powders, sodium bicarbonate, calcium carbonate, alginic acid or sodium arginine, polyoxyethylene sorbitan fatty acid ester, sodium lauryl sulfate, monoglyceride stearate and salts thereof.

Examples of the fillers include the sugars described above and/or calcium phosphate (e.g., tricalcium phosphate and calcium hydrogen phosphate).

Examples of the emulsifiers include sorbitan fatty acid ester, glycerin fatty acid ester, sucrose fatty acid ester, and propylene glycol fatty acid ester.

Examples of the flow modulators and the lubricants include silicate, talc, stearate and polyethylene glycol.

The inhibitor of HCV infection may also contain, in addition to the additives described above, a corrigent, a solubilizing auxiliary (solubilizer), a suspending agent, a diluent, a surfactant, a stabilizer, an absorption promoter (e.g., quaternary ammonium salts and sodium lauryl sulfate), an expander, a humectant (e.g., glycerin and starch), an adsorbent (e.g., starch, lactose, kaolin, bentonite, and colloidal silicic acid), a disintegration inhibitor (e.g., saccharose, stearin, cacao butter, and hydrogenated oil), a coating agent, a colorant, a preservative, an antioxidant, a fragrance, a flavoring agent, a sweetener, a buffer, etc., if necessary.

### (3) Additional Drug

The inhibitor of HCV infection of the present invention can also contain an additional drug without losing the pharmacological effect of the active ingredient. In this context, examples of the “additional drug” include a drug suppressing HCV infection in a mechanism of action similar to that of the anti-occludin antibody and/or the fragment thereof of the first aspect (e.g., an anti-hCD81 antibody), and a drug having a mechanism of action different from that of the anti-occludin antibody and/or the fragment thereof of the first aspect, for example, suppressing growth after persistent HCV infection (e.g., daclatasvir and asunaprevir). Alternatively, the additional drug may be a drug having a pharmacological effect irrelevant to HCV infection. Examples thereof include gastric coating agents and antibiotics.

When the inhibitor of HCV infection of the present invention is a combination formulation containing the additional drug, such a combination formulation can be expected to have synergistic effects such as the polyphonic suppression of HCV infection and is therefore convenient.

### 2-2-2. Dosage Form

The dosage form of the inhibitor of HCV infection of the present invention is not particularly limited as long as the dosage form does not inactivate or hardly inactivates the anti-occludin antibody and/or the fragment thereof serving as an active ingredient and can sufficiently exert its pharmacological effect in vivo after administration.

The dosage form can be classified into a liquid dosage form or a solid dosage form (including a semisolid dosage form such as a gel) according to the form thereof. The inhibitor of HCV infection of the present invention may have any of these dosage forms. Also, the dosage form can be broadly classified into an oral dosage form or a parenteral dosage form according to an administration method. Likewise, the inhibitor of HCV infection of the present invention may have any of these dosage forms.

As specific examples of the dosage form, in the case of the oral dosage form, it includes liquid dosage forms such as suspensions, emulsions, and syrups, and solid dosage forms such as powdered formulations (including dusts, powders, and lozenges), granules, tablets, capsules, sublingual formulations, and troches. Examples of the parenteral dosage form include liquid dosage forms such as injections, suspensions, emulsions, eye drops, and nasal drops, and solid dosage forms such as creams, ointments, plasters, patches, and suppositories. The dosage form is preferably any of the oral dosage forms or a liquid dosage form injection for a parenteral dosage form.

### 2-2-3. Administration Method

Any method known in the art can be applied to the inhibitor of HCV infection of the present invention as long as the method can administer an effective amount of the anti-occludin antibody and/or the fragment thereof of the first aspect serving as an active ingredient to an organism for the prevention of HCV infection.

In the present description, the “effective amount” refers to an amount that is necessary for the active ingredient to exert its functions, i.e., necessary for the inhibitor of HCV infection to inhibit HCV infection in the present invention, and imparts no or little side effect to an organism to receive it. This effective amount may vary depending on information on a subject, an administration route, and the number of doses, etc. The “subject” refers to an animal individual to receive the inhibitor of HCV infection of the present aspect or a vaccine for the inhibition of HCV infection of the third aspect. The subject of the inhibitor of HCV infection of the present invention is a human or a chimpanzee in principle because of the host specificity of HCV. However, a transgenic animal capable of expressing human or chimpanzee CD81 and occludin may be used as the subject. The subject is preferably a human. The “information on a subject” is various pieces of information for individuals on the subject and includes, for example, the age, body weight, sex, general health conditions, drug sensitivity, and the presence or absence of ongoing medication of the subject. The effective amount and a dose to be calculated on the basis of it are determined at a physician's or veterinarian's discretion according to the information on the individual subject, etc. When the administration at a large dose of the inhibitor of HCV infection of the present invention is necessary for producing a sufficient inhibitory effect on HCV infection, the inhibitor of HCV infection may be administered at several divided doses in order to reduce the burden on the subject.

The method for administering the inhibitor of HCV infection of the present invention may be systemic administration or local administration. Examples of the systemic administration include intravascular injection such as intravenous injection, and oral administration. Examples of the local administration include local injection. The active ingredient of the inhibitor of HCV infection of the present invention is the anti-occludin antibody and/or the fragment thereof and is therefore constituted by a peptide. Thus, it is preferred for oral administration to perform appropriate procedures such as use of appropriate DDS (drug delivery system) in order to protect the active ingredient from degradation by a digestive enzyme. Since HCV infection occurs in the liver, it is preferred for local injection to administer the inhibitor of HCV infection of the present invention to the liver which is a site to be treated. Systemic administration by intravascular injection is particularly preferred because this approach is less invasive and can spread the active ingredient throughout the body including the liver.

As one example of the specific dose, the effective amount of the inhibitor of HCV infection per day is in the range of usually 1 to 2000 mg, preferably 1 to 1000 mg, more preferably 1 to 500 mg, for example, for administration to a human adult male (body weight: 60 kg) having the early stage of development of hepatitis C. In the case of administering the inhibitor of HCV infection of the present invention to a subject, the effective dose of the antibody of the present invention serving as an active ingredient is selected in the range of 0.001 to 1000 mg/kg body weight per dose. Alternatively, a dose of 0.01 to 100000 mg/body can be selected per subject. However, the dose is not limited thereto.

The administration timing is not particularly limited, but is preferably before symptoms become severe due to HCV infection, i.e., before development of chronic hepatitis C, because the pharmacological effect of the inhibitor of HCV infection of the present invention is the inhibition of HCV infection. A period after early infection of HCV and before progression to the state of persistent infection is more preferred. The inhibitor of HCV infection may be administered before infection with HCV.

### 3. Vaccine for Inhibition of Hepatitis C Virus Infection

### 3-1. Summary

The third aspect of the present invention provides a vaccine for the inhibition of HCV infection. The vaccine for the inhibition of HCV infection of the present aspect consists of a peptide that consists of a portion of an amino acid sequence constituting the second extracellular domain of occludin and comprises the ALCN epitope shown in SEQ ID NO: 1. The vaccine for the inhibition of HCV infection of the present invention, when administered to a subject, can induce the production of the anti-occludin antibody described in the first aspect within the body of the subject and can thereby prevent the subject from HCV infection.

### 3-2. Configuration

### 3-2-1. Constituent

### (1) Vaccine for Inhibition of HCV Infection

The “vaccine” is a pharmaceutical product that is inoculated to an animal for the prevention of an infectious disease. The vaccinated animal then resists the infectious disease owing to improved acquired immunity against a pathogen causative of the infectious disease. Usually, vaccines utilize a pathogen or a portion thereof, or a toxin. Examples thereof include live pathogens with attenuated pathogenicity or toxicity (live vaccines), dead pathogens that have lost their pathogenicity or toxicity by chemical treatment or the like (inactivated vaccines), and toxoids prepared by inactivating toxins produced by pathogens. However, in the present description, the vaccine for the inhibition of HCV infection utilizes a portion of the virus receptor protein occludin expressed on host cells, i.e., host hepatocytes, to be infected with HCV. Thus, unlike general vaccines, the vaccine for the inhibition of HCV infection of the present invention is derived from a portion of the protein of the host of HCV infection, not derived from the pathogen HCV.

The specific configuration of the vaccine for the inhibition of HCV infection of the present invention is a peptide that contains the ALCN epitope shown in SEQ ID NO: 1 and consists of a portion of the amino acid sequence represented by SEQ ID NO: 4 constituting the second extracellular domain of human or chimpanzee occludin, an amino acid sequence derived from the amino acid sequence represented by SEQ ID NO: 4 by deletions, substitutions or additions of a amino acid or multiple amino acids, or an amino acid sequence having 90% or higher amino acid identity to the amino acid sequence represented by SEQ ID NO: 4. The amino acid length of this peptide is not limited and is preferably 4 to 30 amino acids, more preferably 4 to 20 amino acids, further preferably 6 to 18 amino acids or 8 to 15 amino acids.

Specific examples of the vaccine for the inhibition of HCV infection of the present invention include a peptide consisting of the amino acid sequence represented by SEQ ID NO: 6 and 7. The amino acid sequence represented by SEQ ID NO: 6 is a portion of the second extracellular domain of human occludin and corresponds to an amino acid sequence from positions 214 to 223 in the full-length human occludin. The amino acid sequence represented by SEQ ID NO: 7 comprises a peptide consisting of the amino acid sequence represented by SEQ ID NO: 6 and corresponds to an amino acid sequence from positions 214 to 230 in the full-length human occludin.

### (2) Immunopotentiator

The vaccine for the inhibition of HCV infection of the present invention can be used in combination with an immunopotentiator for the purpose of strengthening the immunological responsiveness of a subject and potentiating the effect of the vaccine.

Specific examples of the immunopotentiator include adjuvants and cytokines.

Many types of adjuvants are known in the art. The vaccine for the inhibition of HCV infection of the present invention may be combined with any of the adjuvants. Specific examples of the adjuvants include Freund complete and/or incomplete adjuvants, vitamin E, Montanide, alum, poly-IC and derivatives thereof (poly-ICLC, etc.), squalene and/or tocopherol, QS-21 derived from Quillaja saponaria saponin, MPL (SmithKline Beecham plc), QS21 (SmithKline Beecham plc), lipopolysaccharides of Salmonella minnesota R595 of the genus Salmonella, MPL (3-desacyl-4′-monophosphoryl lipid A) which is a nontoxic derivative thereof, and QS-7, QS-17, QS-18 and QS-L1 (So H. S., et al., 1997, Molecules and cells, 7: 178-186).

The mixing ratio between the vaccine for the inhibition of HCV infection of the present invention and the adjuvant is not particularly limited and can be in the range of, for example, 1:10 to 10:1, preferably 1:5 to 5:1. The mixing ratio is more preferably 1:1.

The cytokine may be any cytokine having the property of stimulating lymphocytes or antigen-presenting cells. Specific examples of such cytokines include IL-12, IL-18, GM-CSF, IFN-α, IFN-β, IFN-ω, IFN-γ and Flt3.

### (3) Pharmaceutically Acceptable Carrier

The vaccine for the inhibition of HCV infection of the present invention can be used in combination with a pharmaceutically acceptable carrier, if necessary.

The configuration of the pharmaceutically acceptable carrier can be the same as in the pharmaceutically acceptable carrier constituting the inhibitor of HCV infection of the second aspect. For the pharmaceutically acceptable carrier, see the second aspect. Thus, the specific description thereof will be omitted here.

### 3-2-2. Dosage Form

The dosage form of the vaccine for the inhibition of HCV infection of the present invention basically conforms to the dosage form of the inhibitor of HCV infection described in the second aspect. Thus, the specific description thereof will be omitted here. However, for example, an injection of a solution, a suspension or an emulsion is preferred because ordinary vaccines are often administered as a parenteral liquid dosage form, though the dosage form is not limited thereto.

### 3-2-3. Administration Method

The method for administering the vaccine for the inhibition of HCV infection of the present invention can basically conform to the method for administering the inhibitor of HCV infection described in the second aspect. Thus, the specific description thereof will be omitted here.

In the case of administering the immunopotentiator with the vaccine for the inhibition of HCV infection of the present invention, the order of their administration is not limited. For example, the vaccine for the inhibition of HCV infection may be administered to a subject before or after the immunopotentiator or may be administered concurrently with the immunopotentiator.

The vaccine for the inhibition of HCV infection of the present invention may be combined with the immunopotentiator and/or the pharmaceutically acceptable carrier and provided as a kit for the prevention of HCV infection supplemented, if necessary, with a buffer, a syringe, an injection needle, an instruction manual, etc.

### EXAMPLES

### Example 1

### (Object)

An object is to prepare an anti-human occludin monoclonal antibody.

### (Method)

A region that was a portion of the second extracellular domain of human occludin and corresponded to positions 214 to 230 was used as an antigen. The peptide was synthesized on the basis of information on the amino acid sequence (SEQ ID NO: 7: ALCNQFYTPAATGLYVD) (Medical & Biological Laboratories Co., Ltd.). Next, in order to enhance antigenic stimulation, keyhole limpet hemocyanin (KLH) (Medical & Biological Laboratories Co., Ltd.) was linked as a carrier protein to the N terminus.

Antibody preparation was performed by the hybridoma method. The basic method conformed to a routine method. Four special disease mice (Medical & Biological Laboratories Co., Ltd.) were subcutaneously immunized a total of 3 times at 2-week-intervals with 50 μg/shot/mouse of the antigen at a concentration of 1 mg/mL. The spleens were harvested from the individuals thus immunized, and B cells were collected. The collected B cells were fused with mouse myeloma P6 cells to prepare hybridomas. Culture supernatants of the hybridomas were collected and screened for binding activity against the antigenic peptide by ELISA.

### (Results)

As a result, 31 anti-occludin monoclonal antibodies were obtained as positive clones having the ability to bind to the antigen.

### Example 2

### (Object)

An object is to evaluate the anti-occludin monoclonal antibodies obtained in Example 1 for their inhibitory effects on HCVpv infection.

### (Method)

The hybridoma cells producing each antibody obtained in Example 1 were cultured at 37° C. in the presence of 5% CO₂ for 3 days in RPMI (Wako Pure Chemical Industries, Ltd.) medium supplemented with 20% FBS (GIBCO/Thermo Fisher Scientific Inc.) and 1% penicillin/streptomycin (GIBCO/Thermo Fisher Scientific Inc.). The culture supernatant obtained after the culture was used as an anti-occludin antibody-producing clone-containing medium.

A well differentiated human liver cancer-derived cell line Huh7.5.1 cell line having high sensitivity of HCV replication (kindly provided by professor Matsuura from Research Institute for Microbial Diseases (Osaka University)) was used as host cells for HCV infection. The Huh7.5.1 cells were cultured at 37° C. in the presence of 5% CO₂ in D-MEM high Glucose (Sigma-Aldrich Co. LLC) medium supplemented with 10% FBS (GIBCO/Thermo Fisher Scientific Inc.). A 0.25% trypsin-EDTA solution was used in the subculture of the cells.

HCVpv was used as HCV. The HCVpv (pseudotyped HCV) is vesicular stomatitis virus (VSV) bearing HCV envelope proteins and is known as a pseudotyped virus of HCV (Matsuura Y, et al., 2001, Virology, 286: 263-275). In the present description, 3 types of HCVpv were used: coni (genotype: 1a), H77 (genotype: 1b), and 9-3 (genotype: 1b) (kindly provided by professor Matsuura from Research Institute for Microbial Diseases (Osaka University)).

The Huh7.5.1 cells were cultured under the conditions described above in a 48-well flat-bottomed plate (Nunc/Thermo Fisher Scientific Inc.). 24 hours later, 12.5 μL/well of the anti-occludin antibody-producing clone-containing medium, or 20 μg/well (80 μg/mL) of the anti-occludin antibody purified from the anti-occludin antibody-producing clone-containing medium was added to the medium when the medium was replaced. Ab-Rapid PuRe affinity gel (ProteNova Co., Ltd.) was used in the antibody purification.

A mock group supplemented with only a medium was established as a negative control, while a CD81 group supplemented with 1.25 μg/well (5 μg/mL) of an anti-human CD81 antibody (hereinafter, also referred to as an “anti-CD81 antibody”) (JS-81 clone; BD Pharmingen) (Fofana I., et al., 2013, PLoS One, 8: e64221) was established as a positive control. 1 hour later, 15 μL of HCVpv (coni or H77) or 15 μL of GFP having no HCV envelope protein as a negative control was added to each well. 24 hours later, luciferase activity was quantified. Specifically, after removal of the medium, 100 μL of a lysis solution was added to each well. 15 minutes later, a 20 μL aliquot was collected and mixed with 50 μL of a luciferase substrate, and the mixture was vortexed and then assayed using a luminometer. When 90% or more decrease was observed as compared with the mock group, the inhibitory effect on infection was determined to be present.

### (Results)

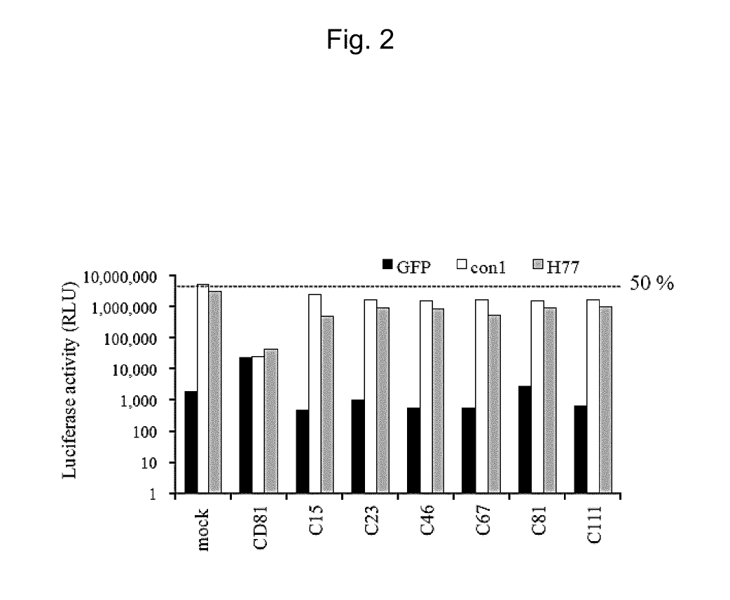

From the 31 clones of anti-occludin antibody candidates obtained in Example 1, 6 clones (C15, C23, C46, C67, C81, and C111) shown in FIG. 2 were obtained as clones exhibiting an infection inhibition rate of 50% or more. On the basis of the results of FIG. 2, the infection inhibition rates of the 6 types of clones and the anti-CD81 antibody were calculated when the infection inhibition rate of the mock was defined as 0%. The results are shown in Table 1.

TABLE 1

| | clone No. | con1 | H77 |
|---|---|---|---|
| αhOCLN | C15 | 56% | 83% |
| candidate | C23 | 69% | 70% |
| | C46 | 70% | 73% |
| | C67 | 68% | 82% |
| | C81 | 71% | 69% |
| | C111 | 69% | 68% |
| positive cont. | αCD81 | 99.8% | 99.1% |

All the infection inhibition rates of the 6 clones were low values as compared with the positive control anti-CD81 antibody (99.8%), though 83% at the maximum was able to be confirmed in the antibody C15.

### Example 3

### (Object)

An object is to evaluate the anti-occludin monoclonal antibodies obtained in Example 1 for their inhibitory effects on HCVcc infection.

### (Method)

Huh7.5.1 cells were used as host cells for HCV infection, as in Example 2. The cells were cultured at 37° C. in the presence of 5% CO₂ in D-MEM high Glucose (Sigma-Aldrich Co. LLC) medium supplemented with 10% FBS (GIBCO/Thermo Fisher Scientific Inc.).

JFH-1 strain-derived HCVcc (cell cultured HCV; genotype: 2a) (Matsuura Y, et al., 2001, Virology, 286: 263-275) established as a laboratory HCV strain (kindly provided by professor Matsuura from Research Institute for Microbial Diseases (Osaka University)) was used as HCV.

500 μL of the medium described above was prepared in each well of a 24-well flat-bottomed plate. The Huh7.5.1 cells were inoculated at 1×10³ cells/well and then incubated for 48 hours. After medium replacement, the anti-occludin antibody-producing clone-containing medium containing each of the 6 clones selected in Example 2 was administered at 25 to 100 μL/well. 1 hour later, 10 μL of HCVcc was administered to each well. 2 hours later, the medium was replaced with a fresh one. 24 hours later, RNA was extracted, and the HCV RNA in the cells was quantified by real-time PCR.

The real-time PCR employed TaqMan(R) One-Step RT-PCR Master Mix Reagents Kit (Toyobo Co., Ltd.) and was performed using Step one real-time PCR system (Thermo Fisher Scientific Inc.) according to the attached protocol. Reverse-transcription reaction was performed at 90° C. for 30 seconds, 61° C. for 20 minutes and 95° C. for 1 minute, and PCR was performed for 45 cycles each involving 95° C. for 15 seconds and 60° C. for 1 minute. The primer pair used in PCR was NSSA Forward (SEQ ID NO: 8) and NSSA Reverse (SEQ ID NO: 9). An oligonucleotide consisting of the nucleotide sequence represented by SEQ ID NO: 10 was used as TaqMan probe. The TaqMan probe was modified at its 5′ end with FAM and at its 3′ end with TAMURA.

A mock group supplemented with only a medium was established as a negative control, while a CD81 group supplemented with 1.25 μg/well of an anti-CD81 antibody (JS-81 clone; BD Pharmingen) was established as a positive control. An IgG group supplemented with 20 μg of normal mouse IgG (hereinafter, also referred to as “IgG”) (Jackson ImmunoResearch Laboratories Inc.) as an antibody for a negative control was also added for the evaluation of an inhibitory effect on HCVpv infection in Table 2 mentioned later.

### (Results)

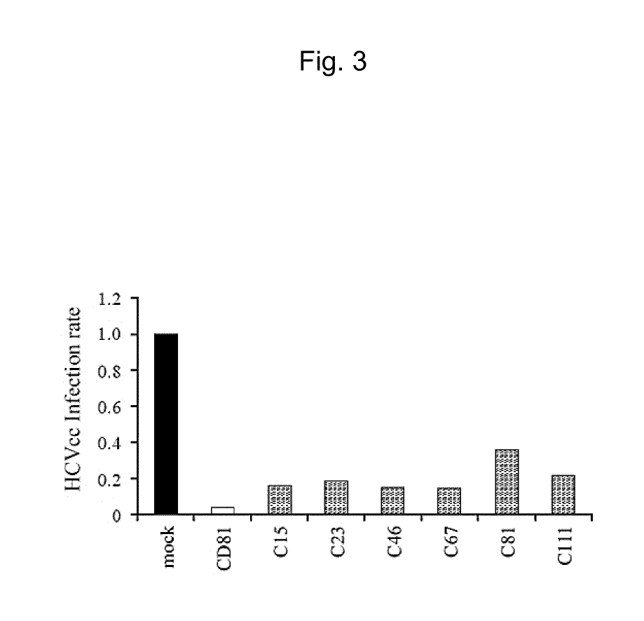

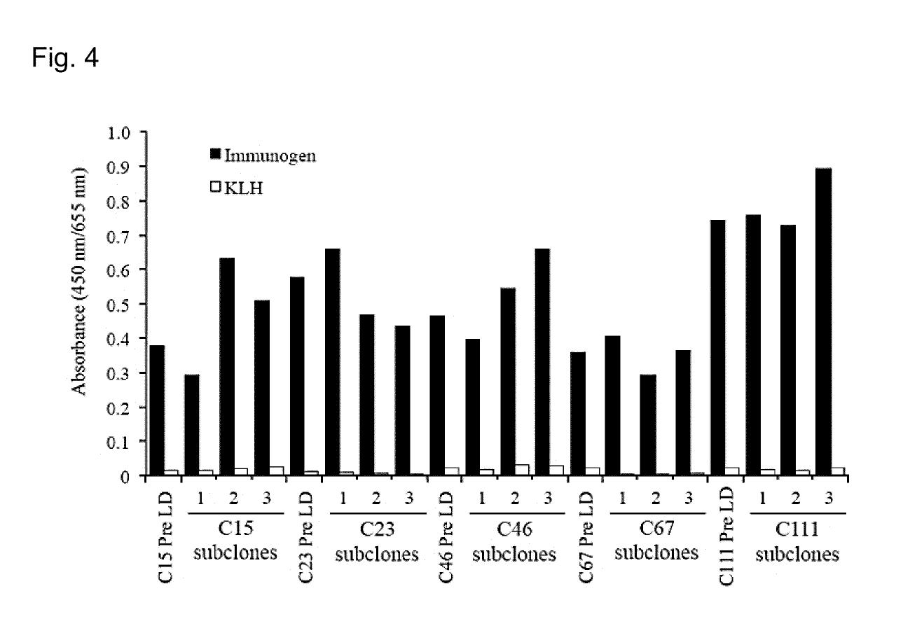

FIG. 3 shows the evaluation results for HCVcc. The HCVcc infection rate on the vertical axis is a relative value when the HCVcc infection rate of the mock group is defined as 1. Among the 6 clones obtained in Example 1, 5 clones showed an infection inhibition rate of 79 to 85%. However, the CD81 antibody had a weak inhibitory effect on infection. Accordingly, a total of 15 clones (subclones) involving 3 clones from each of the 5 clones excluding the CD81 antibody were established by limiting dilution. The anti-occludin monoclonal antibodies contained in culture supernatants were studied for their binding activity against the antigen by ELISA. As a result, as shown in FIG. 4, all the 15 clones showed the ability to bind to the antigen.

Accordingly, purified antibodies of the 15 clones were evaluated for their inhibitory effects on HCVpv infection in the same way as in Example 2. The results are shown in Table 2.

TABLE 2

| | con1 | 9-3 |
|---|---|---|
| C15-1 | 34% | 51% |
| C15-2 | 32% | 31% |
| C15-3 | 11% | 34% |
| C23-1 | 45% | 12% |
| C23-2 | 37% | 52% |
| C23-3 | 41% | 50% |
| C46-1 | 40% | 41% |
| C46-2 | 45% | 44% |
| C46-3 | 25% | 49% |
| C67-1 | 55% | 43% |
| C67-2 | 52% | 51% |
| C67-3 | 31% | 50% |
| C111-1 | 10% | 47% |
| C111-2 | 17% | 41% |
| C111-3 | 15% | 44% |
| CD81 | 99% | 98% |
| IgG | 48% | 43% |

The infection inhibition rate was only 55% for the antibody C67-1, which was largest among the 15 clones, and none of the clones produced a sufficient inhibitory effect as compared with the positive control anti-CD81 antibody (99%).

### Example 4

### (Object)

In Examples 2 and 3, the sufficient inhibitory effects on HCV infection by the anti-occludin antibody of the present invention could not be detected. In the HCVpv and HCVcc evaluation systems of Examples described above, an ordinary monolayer culture system was used. On the presumption that this cell culture method was responsible for the insufficient inhibitory effect on HCV infection, the present inventors reexamined the inhibitory effects on HCV infection of the anti-occludin monoclonal antibodies using a double-chamber culture system which reproduced a hepatocyte environment in living bodies or a route of HCV infection.

### (Method)

The basic operation method conformed to the monolayer culture system described in Example 3 except for a cell culture method and administration methods for antibodies etc. unique to the double-chamber culture system.

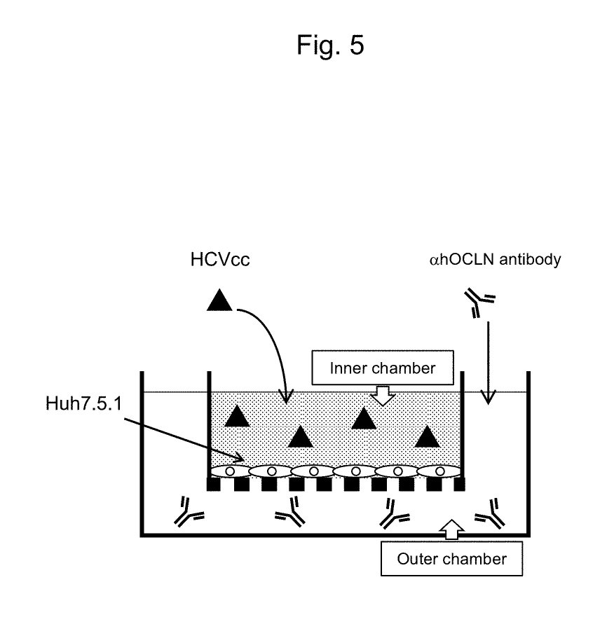

In the double-chamber culture system, a cell culture insert (mixed cellulose ester, 24 wells, a pore size of 0.4 μm, translucent) (BD Falcon) was placed in a 24-well flat-bottomed plate, and 950 μL and 250 μL of a medium were added to the outer chamber and the insert, respectively (FIG. 5). The Huh7.5.1 cells were inoculated at 1×10³ cells into each insert and incubated for 48 hours. After medium replacement, 100 μg each of the anti-occludin monoclonal antibodies of 15 clones used in Example 3 was administered to the outer chamber. As in Example 3, a mock group supplemented with only a medium was established as a negative control, and an IgG group supplemented with 100 μg of IgG (Jackson ImmunoResearch Laboratories Inc.) as an antibody for a negative control was established, while a CD81 group supplemented with 2.5 μg of an anti-CD81 antibody (JS-81 clone; BD Pharmingen) was established as a positive control. IgG or the anti-CD81 antibody was administered to the outer chamber. 1 hour after the antibody or IgG administration, HCVcc (5 μL) for the monolayer culture system was added thereto and incubated for 2 hours, followed by the removal of the medium. The cells were washed twice with a medium. A medium was added thereto again. 24 hours later, RNA was extracted using RNeasy Mini kit (Qiagen N. V.), and the HCV RNA in the cells was quantified by real-time PCR in the same way as in Example 3. In the experiment for inhibition of HCVcc infection, significant difference was tested by the Mann-Whitney U test. The significant difference was regarded as being present at P<0.05.

### (Results)

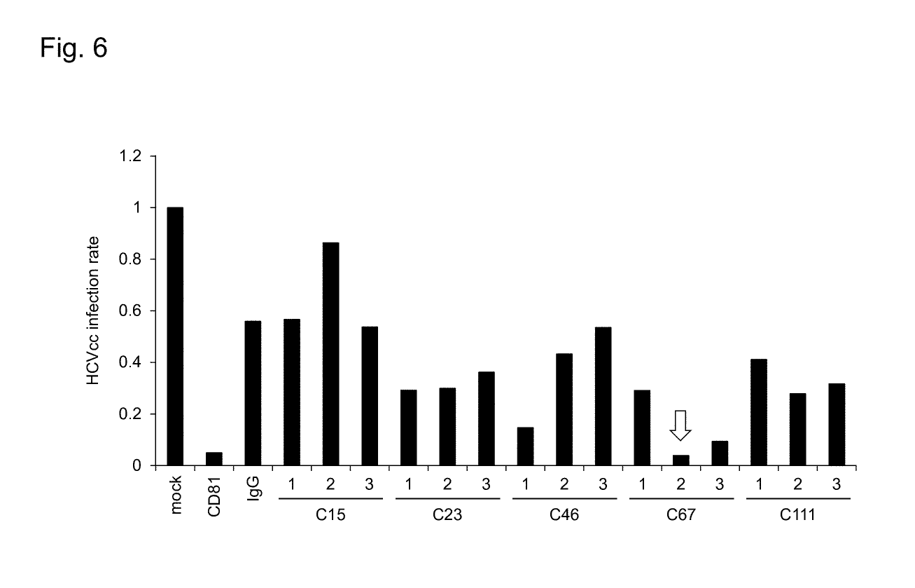

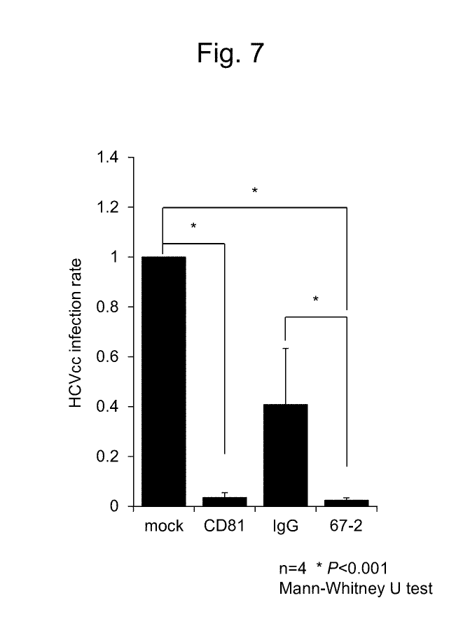

FIG. 6 shows the results of evaluating the inhibition of HCVcc infection by the 15 clones. The HCVcc infection rate on the vertical axis is a relative value when the HCVcc infection rate of the mock group is defined as 1. Among the 15 clones, the 67-2 antibody produced from the hybridoma C67-2 remarkably inhibited HCVcc infection. As shown in FIG. 7, the HCV infection inhibition rate of the 67-2 antibody was 99.6% and was statistically significant with respect to the mock group and the IgG group. Thus, the 67-2 antibody showed an effect equivalent to the CD81 antibody group.

### Example 5

### (Object)

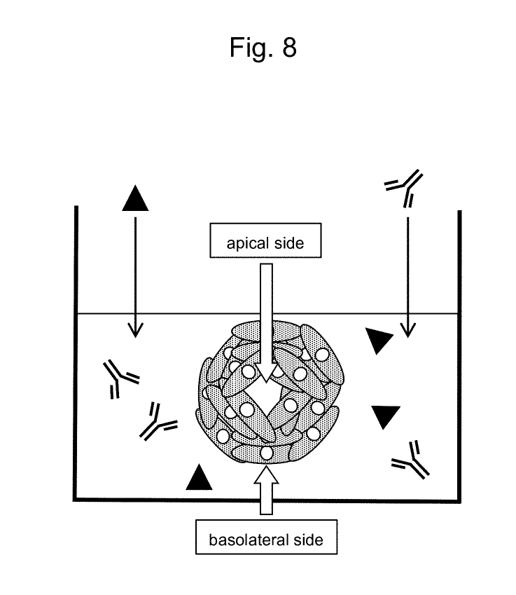

It is known that in a three-dimensional culture system using Matrigel, hepatocyte polarity including a bile capillary-like luminal structure is constructed and reproduced so as to discriminate between an apical (bile capillary) side and a basolateral (sinusoid) side, and this system also has infectiveness with HCV (Furuse M., et al., 1993, J Cell Biol, 123: 1777-1788; and Molina-Jimenez F, et al., 2012, Virology, 425: 31-39) (FIG. 8). Accordingly, an object is to examine the anti-occludin monoclonal antibody for its inhibitory effect on HCV infection using the Matrigel three-dimensional culture system.

### (Method)

In the Matrigel three-dimensional culture system, culture was performed with a modification by the method of Molina-Jimenez et al. (Molina-Jimenez F., et al., 2012, Virology, 425: 31-39.). 50 μL/well of Matrigel (Corning Inc.) was added to a 48-well flat-bottomed plate, and the Huh7.5.1 cells were inoculated at 1×10⁴ cells/well. Then, 200 μL of DMEM medium was added to each well, followed by culture at 37° C. in the presence of 5% CO₂ for 6 days. 50 μg of the 67-2 antibody was administered to each well from the basolateral side. A mock group, a CD81 group supplemented with 2.5 μg of an anti-CD81 antibody, and an IgG group supplemented with 50 μg of normal mouse IgG were established as controls. 1 hour after the administration of the antibody, IgG, etc., HCVcc (10 μL for the monolayer culture system, and 5 μL for the double-chamber and Matrigel culture systems) was added thereto from the basolateral side and incubated for 2 hours, followed by the removal of the medium. The cells were washed twice with a medium. A medium was added thereto again, and the cells were incubated for 24 hours. Then, RNA was extracted from each cell using RNeasy Mini kit (Qiagen N. V.), and the HCV RNA in the cells was quantified by real-time PCR. The other basic operation method conformed to the monolayer culture system described in Example 3. In the experiment for inhibition of HCVcc infection, significant difference was tested by the Mann-Whitney U test. The significant difference was regarded as being present at P<0.05 in all cases.

### (Results)

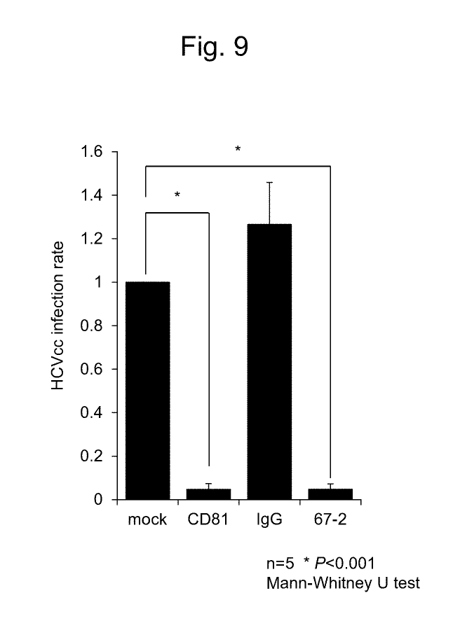

FIG. 9 shows the results of evaluating the 67-2 antibody. The HCVcc infection rate on the vertical axis is a relative value when the HCVcc infection rate of the mock group is defined as 1. As a result, the HCV infection inhibition rate of the 67-2 antibody was 95% and was statistically significant with respect to the mock group. Thus, the 67-2 antibody showed an effect equivalent to the CD81 antibody group.

The results of Example 4 and this Example demonstrated that, for evaluating an inhibitor of HCV infection targeting a molecule localized to tight junction, such as occludin, an ordinary monolayer culture system is not appropriate, and a culture system capable of reproducing hepatocyte polarity and a drug administration route, such as a double-chamber culture system or a Matrigel three-dimensional culture system is necessary.

### Example 6: Cytotoxicity of Anti-Human Occludin Antibody

### (Object)

An object is to examine the anti-occludin monoclonal antibody of the present invention for its cytotoxicity.

### (Method)

The XTT method was used in the evaluation of antibody cytotoxicity. 25 μL/well of Matrigel (Corning Inc.) was added to a 96-well flat-bottomed plate, and the Huh7.5.1 cells were inoculated at 5×10³ cells/well. 100 μL of DMEM medium was added to each well, followed by culture at 37° C. in the presence of 5% CO₂ for 6 days. Only a medium (mock group), 25, 50, and 100 μg/well of the 67-2 antibody, or 25, 50, and 100 μg/well of normal mouse IgG (IgG group) were administered thereto and then incubated for 1 hour.

For the purpose of studying the influence of cytotoxicity on HCV infection at the same time therewith, only a medium (mock group), 50 μg/well of the 67-2 antibody (67-2 group), or 50 μg/well of normal mouse IgG (IgG group) were administered thereto. 1 hour later, 10 μL/well of HCVcc was administered. The cells were further cultured for 2 hours, followed by the evaluation of cytotoxicity. Cell proliferation assay kit II (XTT) (F. Hoffmann-La Roche, Ltd.) was used in analysis, and specific operation conformed to the attached protocol.

### (Results)

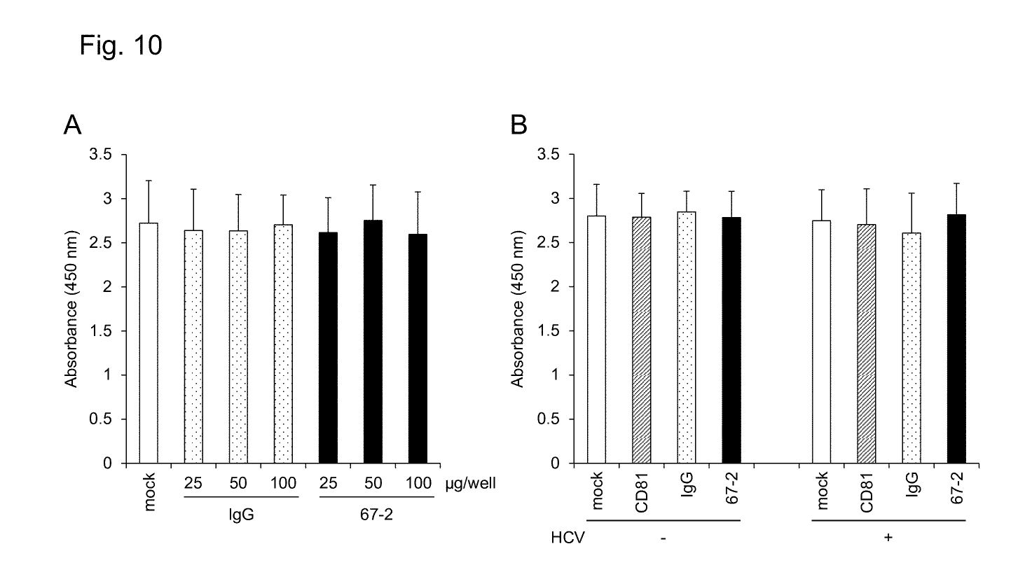

The results are shown in FIG. 10. The 67-2 antibody, which is the anti-occludin monoclonal antibody of the present invention, exhibited no cytotoxicity at any of the concentrations (FIG. 10A). Also, the antibody showed no toxicity to the cells 2 hours after HCVcc infection (FIG. 10B). Thus, the anti-occludin antibody 67-2 antibody was confirmed to be free from severe cytotoxicity at least at concentrations that exert an inhibitory effect on HCV infection, and to exhibit no toxicity to cells.

### Example 7

### (Object)

An object is to conduct isotype analysis and epitope analysis on the 67-2 antibody.

### (Method)

### (1) Antibody Isotype Analysis

The isotype of the 67-2 antibody was analyzed using Iso Strip mouse monoclonal antibody isotyping kit (F. Hoffmann-La Roche, Ltd.). 1 mg/mL of the 67-2 antibody was added dropwise to a development tube and stirred. Then, a strip for isotype was dipped therein. 5 minutes later, the isotype was identified from a blue band detected in the strip portion.

### (2) Epitope Analysis

In Examples 1 to 6, a portion of the second extracellular domain of human occludin was used as an antigen. Thus, the epitope for the 67-2 antibody is supposed to reside within the region corresponding to positions 214 to 230 of occludin and consisting of the amino acid sequence represented by SEQ ID NO: 7.

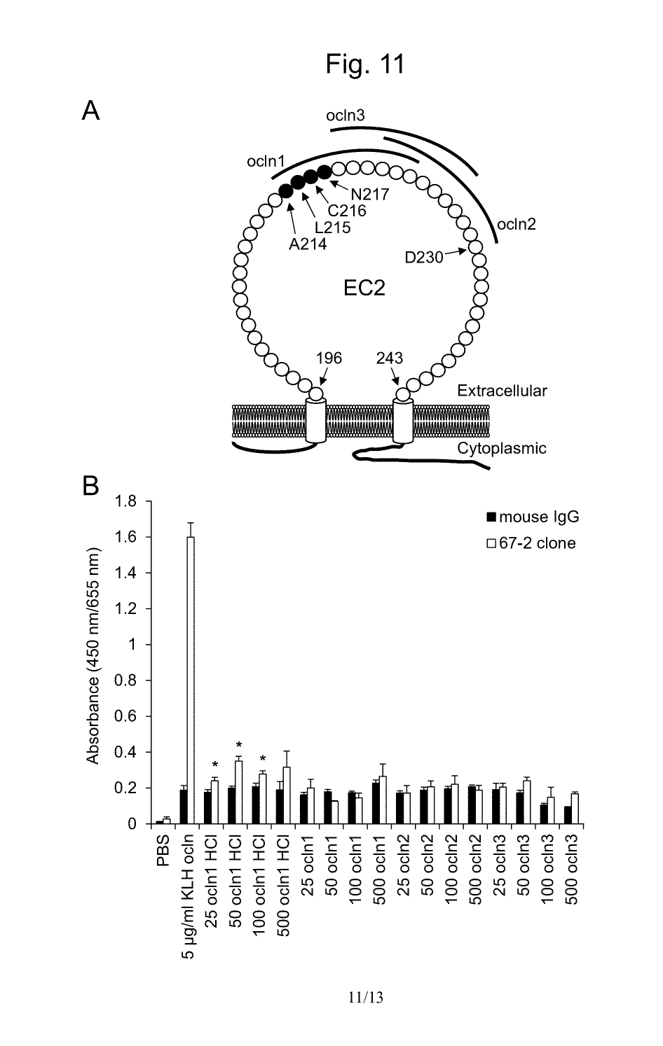

Accordingly, Ocln1 consisting of the amino acid sequence represented by SEQ ID NO: 6 (ALCNQFYTPA) by the truncation of 7 residues from the 3′-terminal side of the amino acid sequence represented by SEQ ID NO: 7, Ocln2 consisting of the amino acid sequence represented by SEQ ID NO: 11 (TPAATGLYVD) by the truncation of 7 residues from the 5′-terminal side thereof, and Ocln3 consisting of the amino acid sequence represented by SEQ ID NO: 12 (QFYTPAATGL) by the truncation of 3 to 4 residues from both terminal sides were prepared as antigenic peptides (FIG. 11A). KLH-Ocln peptide (1 μg/well) was used as a positive control antigen.

Each antigenic peptide was added at 25, 50, 100, and 500 μg/well to an antigen sensitization microplate (Nunc/Thermo Fisher Scientific Inc.) and incubated overnight at room temperature. After blocking with 5% BSA/PBS/0.09% NaN₃ at 37° C. for 2 hours, the 67-2 antibody or normal mouse IgG was added thereto and incubated at room temperature for 1 hour. Ocln1 has a cysteine residue in the sequence and is therefore capable of forming a dimer. Therefore, the analysis was also conducted under acidic conditions (2 N HCl). After washing with PBS, horseradish peroxidase (HRP)-labeled goat anti-mouse IgG (Medical & Biological Laboratories Co., Ltd.) was added thereto as a secondary antibody and incubated at room temperature for 1 hour. After washing with PBS, color reaction was performed with a TMB reagent. The reaction was terminated with 1 M phosphoric acid. The absorbance at 450 nm/655 nm was measured using a microplate reader. Significant difference for the epitope analysis was tested by use of the Student's t test.

### (Results)

### (1) Antibody Isotype Analysis

The blue band detected in the strip portion demonstrated that the 67-2 antibody has an IgG1 H chain and a λ L chain (data not shown).

### (2) Epitope Analysis

FIG. 11B shows the epitope analysis results for the 67-2 antibody. The 67-2 antibody significantly bound only to the Ocln1 peptide within the range of 25, 50, and 100 μg/well under acidic conditions. The absence of reactivity with the Ocln3 peptide suggested that the epitope for the 67-2 antibody is ALCN (SEQ ID NO: 1) which is a portion of the second extracellular domain of human occludin and corresponds to positions 214 to 217.

### Example 8

### (Object)

An object is to examine a mechanism underlying the inhibition of HCV infection by the anti-occludin antibody of the present invention by fluorescent immunostaining.

### (Method)

Fluorescent immunostaining was performed using the 67-2 antibody, which is the anti-occludin monoclonal antibody of the present invention. 50 μL/well of Matrigel (Corning Inc.) was added to an 8-well glass chamber plate, and the Huh7.5.1 cells were inoculated at 5×10³ cells/well. 200 μL of DMEM medium was added to each well, followed by culture at 37° C. in the presence of 5% CO₂ for 6 days. A mock composed of only a medium (−67-2) and 50 μg/well of the 67-2 antibody (+67-2) were combined with the presence or absence of HCVcc administration (+HCV; −HCV) for grouping.

1 hour after the antibody administration, 5 μL/well of HCVcc was administered. 2 hours or 72 hours later, the plate was washed with PBS, followed by fixation in methanol at −20° C. for 10 minutes (2 hr fixation group and 72 hr fixation group). The plate was blocked with 2% BSA/PBS at room temperature for 1 hour.

For the 2 hr later fixation group, a rat anti-occludin antibody (kindly provided by professor Furuse from National Institute for Physiological Sciences) (Saitou M., et al., 1997, Eur J Cell Biol, 73: 222-231) and a rabbit anti-NSSA antibody (kindly provided by professor Matsuura from Research Institute for Microbial Diseases (Osaka University)) (Hamamoto I., et al., 2005, J Virol, 79: 13473-13482) were used as primary antibodies. Fluorescently labeled donkey anti-rat IgG-Alexa Fluor 488 (Molecular Probes, Inc.) was used as a secondary antibody.

For the 72 hr later fixation group, a rabbit anti-NSSA antibody was diluted 200-fold with a signal booster (Beacle, Inc.) and used as a primary antibody. Fluorescently labeled donkey anti-rabbit IgG-Alexa Fluor 488 (Molecular Probes, Inc.) was used as a secondary antibody.

The secondary antibody reaction was performed at room temperature for 1 hour using each antibody diluted 200-fold with PBS. Then, nuclear staining with DAPI was performed at room temperature for 10 minutes, followed by mounting. Observation was conducted under a confocal laser microscope (FV-1000, Olympus Corp.).

### (Results)

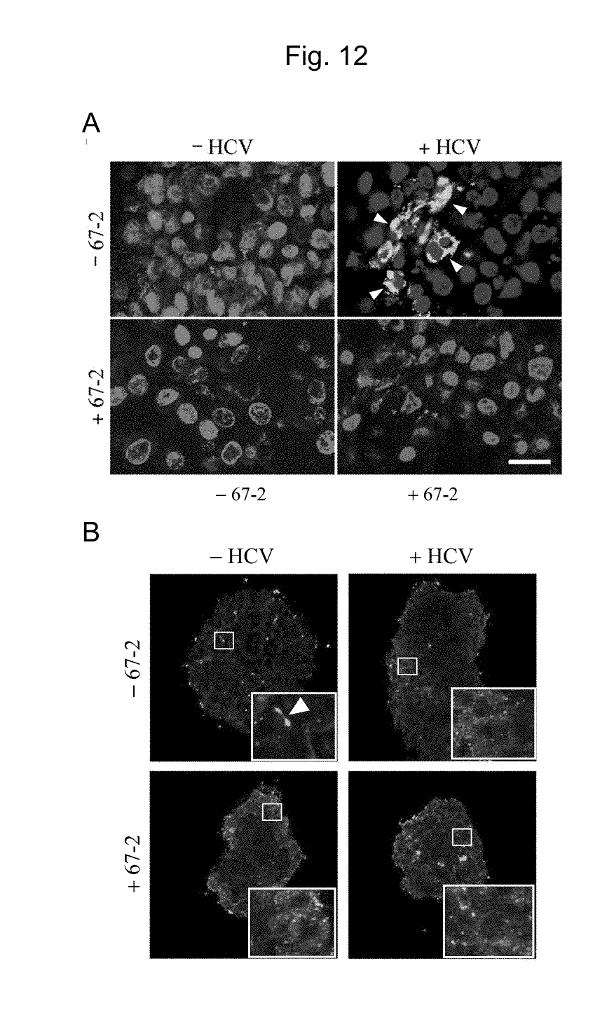

FIG. 12 shows the fluorescent immunostaining results. FIG. 12A shows the results for the 72 hr later fixation group, and FIG. 12B shows results for the 2 hr later fixation group.

As seen in FIG. 12A, approximately 5 to 10% of the cells in the −67-2 group unsupplemented with the 67-2 antibody was NSSA-positive (indicated by arrowheads in the diagram) in the presence of HCVcc (+HCV) and confirmed to have HCV infection. On the other hand, HCV-infected cells were not able to be confirmed in the +67-2 group supplemented with the 67-2 antibody. This suggests that the 67-2 antibody of the present invention also has an inhibitory effect on HCV infection at the protein level.

As seen in FIG. 12B, occludin was localized to the tight junction region (arrowhead) under the −67-2/−HCV conditions. On the other hand, occludin localization was shifted from the tight junction region to the cytoplasm under the +67-2/−HCV and +67-2/+HCV conditions, i.e., under conditions involving the administration of the 67-2 antibody irrespective of the presence or absence of HCV. This result suggests that the 67-2 antibody induces the endocytosis of occludin and evades the interaction with HCV or receptors, thereby inhibiting infection.

### Example 9

### (Object)

An object is to obtain the 67-2 antibody gene.

### (Method)

RNA was extracted as total RNA from the hybridoma C67-2 producing the 67-2 antibody using RNeasy Mini kit (Qiagen N. V.). The specific operation conformed to the attached protocol of the kit. Next, a cDNA library was prepared from the obtained total RNA using Gene Racer Kit (Thermo Fisher Scientific Inc.) according to the attached protocol of the kit. Subsequently, the heavy chain and light chain genes of IgG were each amplified by PCR using the prepared cDNA library as a template, a primer pair of Gene Racer 5′ primer (SEQ ID NO: 29) and mIgG1 3′ primer (SEQ ID NO: 30) or mIgλ 3′ primer (SEQ ID NO: 31), and KOD plus polymerase (Toyobo Co., Ltd.) or Platinum Taq polymerase (Thermo Fisher Scientific Inc.). The obtained amplification products were inserted to pT7 Blue vectors (Novagen/Merck KGaA) to obtain a group of heavy chain gene clones and a group of light chain gene clones. A plurality of clones was selected from each group, and the nucleotide sequence of the insert fragment of each clone was determined according to a routine method.

### (Results)

An identical nucleotide sequence was confirmed from each of the heavy chain gene clones or the light chain gene clones. Therefore, they were determined as the heavy chain and light chain genes of the 67-2 antibody. A nucleotide sequence encoding the variable region of each chain was determined from results of sequence alignment. The nucleotide sequence encoding the heavy chain variable region of the 67-2 antibody is shown in SEQ ID NO: 21, and the nucleotide sequence encoding the light chain variable region is shown in SEQ ID NO: 25. On the basis of the Kabat rule, CDR1, CDR2 and CDR3 were predicted from the nucleotide sequence of each variable region. A region encoding CDR1 of the heavy chain variable region is shown in SEQ ID NO: 22, a region encoding CDR2 is shown in SEQ ID NO: 23, and a region encoding CDR3 is shown in SEQ ID NO: 24. Also, a region encoding CDR1 of the light chain variable region is shown in SEQ ID NO: 26, a region encoding CDR2 is shown in SEQ ID NO: 27, and a region encoding CDR3 is shown in SEQ ID NO: 28.

### Example 10

### (Object)

An object is to establish an anti-occludin humanized antibody-expressing cell line and examine the obtained anti-occludin humanized antibody for its inhibitory effect on HCVcc infection.

### (Method and Results)

### (1) Construction of Heavy Chain and Light Chain Genes of Anti-Occludin Humanized Antibody

Nucleotide sequences encoding CDR1, CDR2, and CDR3 in a human IgG heavy chain gene and a human IgG light chain gene were replaced with the nucleotide sequences encoding the heavy chain CDR1, CDR2, and CDR3 (shown in SEQ ID NOs: 22, 23, and 24, respectively) and the nucleotide sequences encoding the light chain CDR1, CDR2, and CDR3 (shown in SEQ ID NOs: 26, 27, and 28, respectively) of the 67-2 antibody obtained in Example 9 to construct the heavy chain and light chain genes of an anti-occludin humanized antibody.

Subsequently, the heavy chain and light chain genes of the anti-occludin humanized antibody were inserted to expression vectors pEHX1.1 (Toyobo Co., Ltd.) and pELX2.2 (Toyobo Co., Ltd.), respectively, under the control of a promoter to construct a heavy chain expression vector and a light chain expression vector for the anti-occludin humanized antibody.

### (2) Establishment of Anti-Occludin Humanized Antibody-Expressing Cell Line

The two expression vectors for the heavy chain and the light chain of the anti-occludin humanized antibody were introduced to CHO-K1 cells by use of the Lipofectamine method, and transformed clone lines were isolated by limiting dilution. Each clone line was cultured by a routine method, and a culture supernatant containing the anti-occludin humanized antibody was collected. Subsequently, binding activity against the antigenic occludin peptide of SEQ ID NO: 7 was confirmed by ELISA.

As a result, the anti-occludin humanized antibody produced by clone 2D3 line (2D3 antibody) had the strongest antigen binding activity. Accordingly, the clone 2D3 line was established as a CHO-K1 line expressing the anti-occludin humanized antibody.

### (2) Antibody Purification

The 2D3 line mentioned above was cultured by a routine method, and a culture supernatant containing the 2D3 antibody was collected. Subsequently, the culture supernatant was applied onto a protein A-based affinity column (ProteNova Co., Ltd.), followed by the elution of the 2D3 antibody from the column using Gentle Ag/Ab Elution buffer (Thermo Fisher Scientific Inc.). Subsequently, in order to replace the antibody solution with a TB S buffer, the buffer replacement was performed using PD-10 column (GE Healthcare Japan Corp.). The protein concentration in each fraction was measured by the BCA method to determine antibody fractions. The purified antibody was confirmed to be human IgG by SDS-PAGE and Western blot.

### (3) Evaluation of Anti-Occludin Humanized Antibody for its Inhibitory Effect on HCVcc Infection

The anti-occludin humanized antibody 2D3 antibody was examined for its inhibitory effect on HCVcc infection. The basic operation conformed to the method described in Example 3. However, conditions such as a well size were changed as described below. 50 μL/well of Matrigel (Corning Inc.) was added to a 48-well flat-bottomed plate, and the Huh7.5.1 cells were inoculated at 1×10⁴ cells/well. 200 μL of DMEM medium containing 10% FBS was added to each well, followed by culture at 37° C. in the presence of 5% CO₂ for 6 days. 50 μg of the anti-occludin antibody was added to each well. A mock group supplemented with only a medium was established as a negative control, while a CD81 group supplemented with 2.5 μg of an anti-CD81 antibody as a positive control, and an IgG group with 50 μg of normal human IgG added to a TBS buffer were established.

1 hour after the addition of the anti-CD81 antibody, the 2D3 antibody or IgG, 5 μL of HCVcc was added to each well and incubated for 2 hours, followed by the removal of the medium. The cells were washed twice with a medium. Then, a medium was added thereto again, and the cells were incubated for 24 hours. Then, RNA was extracted using RNeasy Mini kit (Qiagen N. V.), and the HCV RNA in the cells was quantified by real-time PCR.

Reverse-transcription reaction and PCR employed RNA-direct(R) Real-time PCR Master Mix Reagents Kit (Toyobo Co., Ltd.) and were performed using Step one real-time PCR system (Thermo Fisher Scientific Inc.) according to the attached protocol of the kit. The reverse-transcription reaction was performed at 90° C. for 30 seconds, 61° C. for 20 minutes and 95° C. for 1 minute, and the PCR reaction was performed for 45 cycles each involving 95° C. for 15 seconds and 60° C. for 1 minute. NSSA Forward primer consisting of the nucleotide sequence represented by SEQ ID NO: 8 and NSSA Reverse primer consisting of the nucleotide sequence represented by SEQ ID NO: 9 were used as a real-time PCR primer pair. A probe consisting of the nucleotide sequence represented by SEQ ID NO: 10 was used as TaqMan probe. The TaqMan probe is modified at its 5′ end with FAM and at its 3′ end with TAMURA.

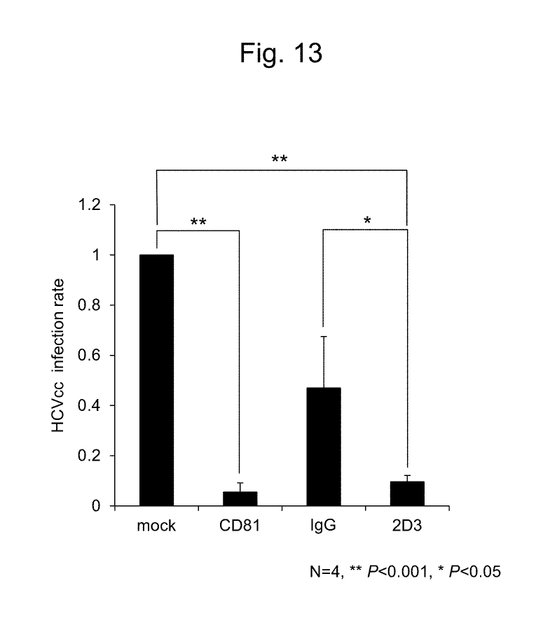

FIG. 13 shows the results of evaluating the inhibitory effect on HCVcc infection of the 2D3 antibody. The HCVcc infection rate on the vertical axis is a relative value when the HCVcc infection rate of the mock group is defined as 1. The 2D3 antibody remarkably inhibited HCVcc infection, as with the positive control anti-CD81 antibody. The HCV infection inhibition rate of the 2D3 antibody was approximately 90% and was a statistically significantly low infection rate with respect to the mock group and the IgG group. This result suggests that the anti-occludin humanized antibody of the present invention has an inhibitory effect on HCV infection.

1. An anti-occludin antibody binding to a peptide which consists of a portion of an amino acid sequence constituting a second extracellular domain of occludin and contains the amino acid sequence represented by SEQ ID NO: 1 as an epitope.

2. The anti-occludin antibody according to claim 1, wherein the second extracellular domain of occludin consists of any of the following amino acid sequences:
(a) the amino acid sequence represented by SEQ ID NO: 4,
(b) an amino acid sequence derived from the amino acid sequence represented by SEQ ID NO: 4 by deletions, substitutions or additions of an amino acid or multiple amino acids, and
(c) an amino acid sequence having 90% or higher amino acid identity to the amino acid sequence represented by SEQ ID NO: 4.

3. The anti-occludin antibody according to claim 1, wherein the peptide consists of the amino acid sequence represented by SEQ ID NO: 6 or 7.

4. The anti-occludin antibody according to claim 1, wherein the antibody is a monoclonal antibody.

5. The anti-occludin antibody according to claim 4, wherein the monoclonal antibody comprises
a heavy chain variable region comprising
CDR1 consisting of the amino acid sequence represented by SEQ ID NO: 14,
CDR2 consisting of the amino acid sequence represented by SEQ ID NO: 15, and
CDR3 consisting of the amino acid sequence represented by SEQ ID NO: 16, and a light chain variable region comprising
CDR1 consisting of the amino acid sequence represented by SEQ ID NO: 18,
CDR2 consisting of the amino acid sequence represented by SEQ ID NO: 19, and
CDR3 consisting of the amino acid sequence represented by SEQ ID NO: 20.

6. The anti-occludin antibody according to claim 4, wherein the monoclonal antibody comprises
a heavy chain variable region consisting of the amino acid sequence represented by SEQ ID NO: 13, and
a light chain variable region consisting of the amino acid sequence represented by SEQ ID NO: 17.

7. The anti-occludin antibody according to claim 4, wherein the antibody is a humanized antibody, a chimeric antibody, a single-chain antibody, or a multispecific antibody.

8. A fragment of an anti-occludin antibody according to claim 1, wherein the fragment retains binding activity to the epitope.

9. An inhibitor of hepatitis C virus infection, comprising an anti-occludin antibody according to at least claim 1 and/or an anti-occludin antibody fragment according to claim 8 as an active ingredient.

10. A vaccine for the inhibition of hepatitis C virus infection, comprising a peptide which consists of a portion of an amino acid sequence constituting a second extracellular domain of occludin and contains the amino acid sequence represented by SEQ ID NO: 1 as an epitope.

11. The vaccine for the inhibition of hepatitis C virus infection according to claim 10, wherein the second extracellular domain of occludin consists of any of the following amino acid sequences:
(a) the amino acid sequence represented by SEQ ID NO: 4,
(b) an amino acid sequence derived from the amino acid sequence represented by SEQ ID NO: 4 by deletions, substitutions or additions of an amino acid or multiple amino acids, and
(c) an amino acid sequence having 90% or higher amino acid identity to the amino acid sequence represented by SEQ ID NO: 4.

12. The vaccine for the inhibition of hepatitis C virus infection according to claim 10, wherein the peptide consists of 4 to 20 amino acids.

13. The vaccine for the inhibition of hepatitis C virus infection according to claim 12, wherein the peptide consists of the amino acid represented by SEQ ID NO: 6 or 7.


SEQUENCE LISTING (31 sequences)

SEQ ID NO: 1 - protein, 4 aa, Homo sapiens
  epitope
ALCN

SEQ ID NO: 2 - protein, 522 aa, Homo sapiens
  occludin
MSSRPLESPPPYRPDEFKPNHYAPSNDIYGGEMHVRPMLSQPAYSFYPEDEILHFYKWTSPPGVIRILSMLIIVMCIAIFACVASTLAWDRGYGTSLLGGSVGYPYGGSGFGSYGSGYGYGYGYGYGYGGYTDPRAAKGFMLAMAAFCFIAALVIFVTSVIRSEMSRTRRYYLSVIIVSAILGIMVFIATIVYIMGVNPTAQSSGSLYGSQIYALCNQFYTPAATGLYVDQYLYHYCVVDPQEAIAIVLGFMIIVAFALIIFFAVKTRRKMDRYDKSNILWDKEHIYDEQPPNVEEWVKNVSAGTQDVPSPPSDYVERVDSPMAYSSNGKVNDKRFYPESSYKSTPVPEVVQELPLTSPVDDFRQPRYSSGGNFETPSKRAPAKGRAGRSKRTEQDHYETDYTTGGESCDELEEDWIREYPPITSDQQRQLYKRNFDTGLQEYKSLQSELDEINKELSRLDKELDDYREESEEYMAAADEYNRLKQVKGSADYKSKKNHCKQLKSKLSHIKKMVGDYDRQKT

SEQ ID NO: 3 - protein, 48 aa, Homo sapiens
  occludin 2nd ectodomain
GVNPTAQSSGSLYGSQIYALCNQFYTPAATGLYVDQYLYHYCVVDPQE

SEQ ID NO: 4 - protein, 48 aa, Homo sapiens; identical to SEQ ID NO: 3
  occludin 2nd ectodomain

SEQ ID NO: 5 - protein, 508 aa, Homo sapiens
  occludin
MSSRPLESPPPYRPDEFKPNHYAPSNDIYGGEMHVRPMLSQPAYSFYPEDEILHFYKWTSPPGVIRILSMLIIVMCIAIFACVASTLAWDRGYGTSLLGGSVGYPYGGSGFGSYGSGYGYGYGYGYGYGGYTDPRAAKGFMLAMAAFCFIAALVIFVTSVIRSEMSRTRRYYLSVIIVSAILGIMVFIATIVYIMGVNPTAQSSGSLYGSQIYALCNQFYTPAATGLYVDQYLYHYCVVDPQEAIAIVLGFMIIVAFALIIFFAVKTRRKMDRYDKSNILWDKEHIYDEQPPNVEEWVKNVSAGTQDVPSPPSDYVERVDSPMAYSSNGKVNDKRFYPESSYKSTPVPEVVQELPLTSPVDDFRQPRYSSSGNFETPSKRAPAKGRAGRSKRTEQDHYETDYTTGGESCDELEEDWIREYPPITSDQQRQLYKRNFDTGLQEYKSLQSELDEINKELSRLDKELDDYREESEEYMSADYKSKKNHCKQLKSKLSHIKKMVGDYDRQKT

SEQ ID NO: 6 - protein, 10 aa, Homo sapiens
  ocln1occludin position 214-223
ALCNQFYTPA

SEQ ID NO: 7 - protein, 17 aa, Homo sapiens
  occludin position 214-230
ALCNQFYTPAATGLYVD

SEQ ID NO: 8 - DNA, 19 bp, Artificial
  primer NS5A Forward
agtaccacaaggcctttcg

SEQ ID NO: 9 - DNA, 17 bp, Artificial
  primer NS5A Reverse
cgggagagccatagtgg

SEQ ID NO: 10 - DNA, 21 bp, Artificial
  Taqman probe
ctgcggaaccggtgagtacac

SEQ ID NO: 11 - protein, 10 aa, Homo sapiens
  ocln2 occludin position 221-230
TPAATGLYVD

SEQ ID NO: 12 - protein, 9 aa, Homo sapiens
  ocln3 occludin position 218-226
QFYTPAATG

SEQ ID NO: 13 - protein, 138 aa, Mus musculus
  heavy chain variable domain of 67-2 clone
MGWSWIFLFLLSGTAGVLSEVQLQQSGPELVKPGASVKISCKASGYTFTDYIMDWVKQSHGKSLEWIGYIYPNNGGTGYNQKFKSKATLTIDKSSSTAYMELHSLTSEDSAVYYCAREDGDYGYFDVWGAGTTVTVSS

SEQ ID NO: 14 - protein, 5 aa, Mus musculus
  heavy chain CDR1 of 67-2 clone
DYIMD

SEQ ID NO: 15 - protein, 17 aa, Mus musculus
  heavy chain CDR2 of 67-2 clone
YIYPNNGGTGYNQKFKS

SEQ ID NO: 16 - protein, 10 aa, Mus musculus
  heavy chain CDR3 of 67-2 clone
EDGDYGYFDV

SEQ ID NO: 17 - protein, 129 aa, Mus musculus
  light chain variable domain of 67-2 clone
MAWTSLILSLLALCSGASSQAVVTQESALTTSPGGTVILTCRSSTGAVTTSNYANWVQEKPDHLFTGLIGGTSNRAPGVPVRFSGSLIGDKAALTITGAQTEDDAMYFCALWYSTHYVFGGGTKVTVLG

SEQ ID NO: 18 - protein, 14 aa, Mus musculus
  light chain CDR1 of 67-2 clone
RSSTGAVTTSNYAN

SEQ ID NO: 19 - protein, 7 aa, Mus musculus
  light chain CDR2 of 67-2 clone
GTSNRAP

SEQ ID NO: 20 - protein, 9 aa, Mus musculus
  light chain CDR3 of 67-2 clone
ALWYSTHYV

SEQ ID NO: 21 - DNA, 414 bp, Mus musculus
  heavy chain variable region of 67-2 clone
atgggatggagctggatctttctcttcctcctgtcaggaactgcaggtgtcctctctgaggtccagctgcagcagtctggacctgaactggtgaagcctggggcttcagtgaagatatcctgcaaggcttctggatacacattcactgactacatcatggactgggtgaagcagagccatggaaagagccttgagtggattggatatatttatcctaataatggtggtactggctacaaccagaagttcaagagcaaggccacattgactatagacaagtcctccagcacagcctacatggagctccacagcctgacatctgaggactctgcagtctattactgtgcaagagaagatggtgactacgggtacttcgatgtctggggcgcagggaccacggtcaccgtctcctca

SEQ ID NO: 22 - DNA, 15 bp, Mus musculus
  heavy chain CDR1 of 67-2 clone
gactacatcatggac

SEQ ID NO: 23 - DNA, 51 bp, Mus musculus
  heavy chain CDR2 of 67-2 clone
tatatttatcctaataatggtggtactggctacaaccagaagttcaagagc

SEQ ID NO: 24 - DNA, 30 bp, Mus musculus
  heavy chain CDR3 of 67-2 clone
gaagatggtgactacgggtacttcgatgtc

SEQ ID NO: 25 - DNA, 387 bp, Mus musculus
  light chain variable region of 67-2 clone
atggcctggacttcacttatactctctctcctggctctctgctcaggagccagttcccaggctgttgtgactcaggaatctgcactcaccacatcacctggtggaacagtcatactcacttgtcgctcaagtactggggctgttacaactagtaactatgccaactgggtccaagaaaaaccagatcatttattcactggtctaataggtggtaccagcaaccgagctccaggtgttcctgtcagattttcaggctccctgattggagacaaggctgccctcaccatcacaggggcacagactgaggatgatgcaatgtatttctgtgctctatggtacagcacccattatgttttcggcggtggaaccaaggtcactgtcctaggt

SEQ ID NO: 26 - DNA, 43 bp, Mus musculus
  light chain CDR1 of 67-2 clone
tcgctcaagtactggggctgttacaactagtaactatgccaac

SEQ ID NO: 27 - DNA, 21 bp, Mus musculus
  light chain CDR2 of 67-2 clone
ggtaccagcaaccgagctcca

SEQ ID NO: 28 - DNA, 27 bp, Mus musculus
  light chain CDR3 of 67-2 clone
gctctatggtacagcacccattatgtt

SEQ ID NO: 29 - DNA, 23 bp, Artificial
  Gene Racer 5′primer
cgactggagcacgaggacactga

SEQ ID NO: 30 - DNA, 28 bp, Artificial
  mIgG1 3′primer
ggatccaattttcttgtccaccttggtg

SEQ ID NO: 31 - DNA, 20 bp, Artificial
  mIglambda 3′primer
ccagtgtggctttgttttcc